(19) United States
(12) Patent Application Publication
(10) Pub. No.: US 2017/0087166 A1
(43) Pub. Date: Mar. 30, 2017

(54) METHODS FOR TREATING AND PREVENTING ALLERGIES AND ACUTE ALLERGIC RESPONSES

(71) Applicant: National Jewish Health, Denver, CO (US)

(72) Inventors: Erwin Gelfand, Englewood, CO (US); Michaela Schedel, Denver, CO (US)

(21) Appl. No.: 15/281,642

(22) Filed: Sep. 30, 2016

Related U.S. Application Data
(60) Provisional application No. 62/235,251, filed on Sep. 30, 2015.

(51) Int. Cl. A61K 31/593; C12N 15/113; A61K 31/713; A61K 45/06

(52) U.S. Cl. CPC A61K 31/593; A61K 45/06; C12N 15/1137; A61K 31/713; C12N 2310/14; C12N 2310/531; C12N 2320/31

(57) ABSTRACT

The present invention relates to methods and compositions for treating and/or preventing allergic diseases and/or conditions, as well as for preventing acute allergic responses by administering a therapeutically effective amount of 1,25-dihydroxy vitamin D3 (1,25D3).

### CROSS-REFERENCE TO RELATED APPLICATIONS

This application claims the benefit of priority under 35 U.S.C. §119(e) to U.S. Provisional Patent Application No. 62/235,251, filed Sep. 30, 2015. The entire disclosure of U.S. Provisional Patent Application No. 62/235,251 is incorporated herein by reference.

### GOVERNMENT RIGHTS

This invention was made with government support under grant numbers R01 AI077609 and P01 HL036577 received from the National Institutes of Health. The government has certain rights in the invention.

### REFERENCE TO A SEQUENCE LISTING

This application contains a Sequence Listing submitted electronically as a text file by EFS-Web. The text file, named “2879-195_ST25”, has a size in bytes of 5 KB, and was recorded on 30 Sep. 2016. The information contained in the text file is incorporated herein by reference in its entirety pursuant to 37 CFR §1.52(e)(5).

### BACKGROUND

For a large percentage of asthmatics, inhaled corticosteroids (ICS) are the most effective first-line treatment to control airway inflammation and symptoms in persistent asthma, but an estimated 40% of asthmatics who fail to respond to corticosteroid show no improvement in airway function (Martin, R. J., et al. The Predicting Response to Inhaled Corticosteroid Efficacy (PRICE) trial. J. Allergy Clin. Immunol. 119, 73-80 (2007)) hence, steroid-refractory asthma remains a clinical challenge. An important role for type 2 (Tc2) CD8⁺ T cells in the development of experimental asthma (Hamelmann, E., et al. Requirement for CD8⁺ T cells in the development of airway hyperresponsiveness in a murine model of airway sensitization. J. Exp. Med. 183, 1719-1729 (1996); Isogai, S., et al. CD8⁺ alphabeta T cells can mediate late airway responses and airway eosinophilia in rats. J. Allergy Clin. Immunol. 114, 1345-1352 (2004); Jia, Y., et al. Steroidogenic enzyme CYP11A1 regulates type 2 CD8⁺ T cell skewing in allergic lung disease. Proc. Natl. Acad. Sci. USA 110, 8152-8157 (2013); Miyahara, N., et al. Leukotriene B4 release from mast cells in IgE-mediated airway hyperresponsiveness and inflammation. Am. J. Respir. Cell Mol. Biol. 40, 672-682 (2009); Miyahara, N., et al. Effector CD8⁺ T cells mediate inflammation and airway hyper-responsiveness. Nat. Med. 10, 865-869 (2004); Miyahara, N., et al. Contribution of antigen-primed CD8⁺ T cells to the development of airway hyperresponsiveness and inflammation is associated with IL-13. J. Immunol. 172, 2549-2558 (2004); Miyahara, N., et al. Requirement for leukotriene B4 receptor 1 in allergen-induced airway hyperresponsiveness. Am. J. Respir. Crit. Care Med. 172, 161-167 (2005); Taube, C., et al. The leukotriene B4 receptor (BLT1) is required for effector CD8⁺ T cell-mediated, mast cell-dependent airway hyperresponsiveness. J. Immunol. 176, 3157-3164 (2006)) as a result of their activation by IL-4-producing CD4⁺ T cells (Koya, T., et al. CD8⁺ T cell-mediated airway hyperresponsiveness and inflammation is dependent on CD4⁺IL-4⁺ T cells. J. Immunol. 179, 2787-2796 (2007)) has been demonstrated. In humans, increased numbers of CD8⁺ T cells, which are more resistant than CD4⁺ T cells to corticosteroids (Li, L. B., et al. ATF2 impairs glucocorticoid receptor-mediated transactivation in human CD8⁺ T cells. Blood 110, 1570-1577 (2007)), have been detected in steroid-refractory asthmatics (Gelfand, E. W. & Dakhama, A. CD8⁺ T lymphocytes and leukotriene B4: novel internnovel interactions in the persistence and progression of asthma. J. Allergy Clin. Immunol. 117, 577-582 (2006)) and correlated with lower lung function and reticular basement membrane thickening (van Rensen, E. L., et al. Bronchial CD8 cell infiltrate and lung function decline in asthma. Am. J. Respir. Crit. Care Med. 172, 837-841 (2005)).

Over the last decade, deficiency in vitamin D, a member of the steroid family, has been associated with various inflammatory diseases (Cantorna, M. T., et al. Vitamin D status, 1,25-dihydroxyvitamin D3, and the immune system. Am. J. Clin. Nutr. 80, 1717S-1720S (2004); Correale, J., et al. Gender differences in 1,25 dihydroxyvitamin D3 immunomodulatory effects in multiple sclerosis patients and healthy subjects. J. Immunol. 185, 4948-4958 (2010); Munger, K. L., et al. Vitamin D intake and incidence of multiple sclerosis. Neurology 62, 60-65 (2004); Zhu, Y., et al. Calcium and 1 alpha,25-dihydroxyvitamin D3 target the TNF-alpha pathway to suppress experimental inflammatory bowel disease. Eur. J. Immunol. 35, 217-224 (2005)) including steroid-refractory asthma (Gupta, A., et al. Defective IL-10 expression and in vitro steroid-induced IL-17A in paediatric severe therapy-resistant asthma. Thorax 69, 508-515 (2014); Gupta, A., et al. Relationship between serum Vitamin D, disease severity, and airway remodeling in children with asthma. Am. J. Respir. Crit. Care Med. 184, 1342-1349 (2011)). An association between lower levels of vitamin D and increased asthma severity, reduced lung function, and poor asthma control has been suggested (Gupta, A., et al. Relationship between serum Vitamin D, disease severity, and airway remodeling in children with asthma. Am. J. Respir. Crit. Care Med. 184, 1342-1349 (2011); Brehm, J. M., et al. Serum Vitamin D levels and severe asthma exacerbations in the Childhood Asthma Management Program study. J. Allergy Clin. Immunol. 126, 52-58 e55 (2010); Goleva, E., et al. Steroid requirements and immune associations with Vitamin D are stronger in children than adults with asthma. J. Allergy Clin. Immunol. 129, 1243-1251 (2012); Korn, S., et al. Severe and uncontrolled adult asthma is associated with Vitamin D insufficiency and deficiency. Respir. Res. 14, 25 (2013); Salas, N. M., et al. Vitamin D deficiency and adult asthma exacerbations. J. Asthma 51, 950-955 (2014); Searing, D. A., et al. Decreased serum Vitamin D levels in children with asthma are associated with increased corticosteroid use. J. Allergy Clin. Immunol. 125, 995-1000 (2010); Sutherland, E. R., et al. Vitamin D levels, lung function, and steroid response in adult asthma. Am. J. Respir. Crit. Care Med. 181, 699-704 (2010)). However, it is unclear if vitamin D supplementation impacts the disease as seen in a recent trial in asthmatics (Castro, M., et al. Effect of Vitamin D3 on asthma treatment failures in adults with symptomatic asthma and lower Vitamin D levels: the VIDA randomized clinical trial. J. Am. Med. Association 311, 2083-2091 (2014)) and a potential mechanism of action remains unknown.

Previously, CYP11A1 was identified as an essential component of a novel, pro-allergic mechanistic axis in the development of experimental asthma (CD8⁺ cells) (Jia, Y., et al. Steroidogenic enzyme CYP11A1 regulates type 2 CD8⁺ T cell skewing in allergic lung disease. Proc. Natl. Acad. Sci. USA 110, 8152-8157 (2013); Jia, Y., et al. Stepwise epigenetic and phenotypic alterations poise CD8⁺ T cells to mediate airway hyperresponsiveness and inflammation. J. Immunol. 190, 4056-4065 (2013)) and peanut-induced allergy (CD4⁺ T cells) (Wang, M., et al. The steroidogenic enzyme CYP11A1 is essential for development of peanut-induced intestinal anaphylaxis. J. Allergy Clin. Immunol. 132, 1174-1183 e1178 (2013)). CYP11A1, a mitochondrial P450 cytochrome, is the first and rate-limiting enzyme in steroidogenesis converting cholesterol to pregnenolone (Lavoie, H. A. & King, S. R. Transcriptional regulation of steroidogenic genes: STARD1, CYP11A1 and HSD3B. Exp. Biol. Med. (Maywood) 234, 880-907 (2009)). In the presence of IL-4, CYP11A1 was identified as a critical regulator of CD8⁺ T cell conversion. Together with antigen receptor signaling of differentiated CD8⁺ T cells, CYP11A1 activation was essential for increased IL-13 and decreased IFN-γ production (Jia, Y., et al. Steroidogenic enzyme CYP11A1 regulates type 2 CD8⁺ T cell skewing in allergic lung disease. Proc. Natl. Acad. Sci. USA 110, 8152-8157 (2013); Jia, Y., et al. Stepwise epigenetic and phenotypic alterations poise CD8⁺ T cells to mediate airway hyperresponsiveness and inflammation. J. Immunol. 190, 4056-4065 (2013)). These data linked for the first time steroidogenesis in CD8⁺T cells, a non-classical steroidogenic tissue, to a pro-allergic differentiation pathway.

The inventors demonstrate herein the in vitro and in vivo role of 1,25-dihydroxy vitamin D3 (1,25D3) as a key modulator of the functional conversion of CD8⁺ T cells from IFN-γ- to IL-13-producing cells via a mechanistic link to CYP11A1 activity. This effect appears driven by 1,25D3-mediated changes in the recruitment of the VDR transcription factor to the promoter region of Cyp11a1 paralleled by changes in the enzymatic activation of CYP11A1 and the prevention of lung allergic responses. An epistasic effect between genetic variants in CYP11A1 and VDR is implicated in humans due to protective effects on the development of asthma.

### SUMMARY OF THE INVENTION

One embodiment of the invention relates to a method of treating or preventing an allergic disease in a subject who has, or is at risk of developing an allergic disease, comprising administering to the subject a therapeutically effective amount of 1,25-dihydroxy vitamin D3 (1,25D3).

Another embodiment of the invention relates to a method of preventing an acute allergic response in a subject in need thereof comprising administering to the subject a therapeutically effective amount of 1,25-dihydroxy vitamin D3 (1,25D3).

In one aspect related to any of the embodiments described herein, the allergic disease is selected from asthma, food allergy, an allergic lung disease, allergen-induced airway hyperresponsiveness, allergen-induced inflammation, rhinitis, allergic rhinitis, eosinophilic esophagitis, chronic urticaria, atopic dermatitis, occupational allergy, allergic conjunctivitis, hay fever, airborne allergic sensitivities, stinging insect allergy, hypersensitivity pneumonitis, eosinophilic lung diseases, inflammatory bowel disease, ulcerative colitis, and Crohn's disease.

In one aspect related to any of the embodiments described herein, the allergic disease is caused by one or more proteinaceous allergens.

In one aspect related to any of the embodiments described herein, the subject has been sensitized to an allergen or is at risk of becoming exposed to an allergen.

In one aspect related to any of the embodiments described herein, the food allergy is peanut allergy.

In one aspect related to any of the embodiments described herein, the allergic disease is steroid-refractive asthma.

In one aspect related to any of the embodiments described herein, the method further comprises administering a therapeutically effective amount of a steroidogenic pathway inhibitor. In another aspect, the steroidogenic pathway inhibitor inhibits cytochrome P450 family 11 subfamily A polypeptide 1 (Cyp11A1). In still another aspect, the steroidogenic pathway inhibitor is aminoglutethamide or a Cyp11A1 siRNA or shRNA molecule. In still yet another aspect, the steroidogenic pathway inhibitor is trilostane or metyrapone.

In one aspect related to any of the embodiments described herein, the subject is a human.

### BRIEF DESCRIPTION OF THE DRAWINGS

FIG. 1 shows IFN-γ and IL-13 expression in CD8⁺ T cells differentiated in IL-2 or IL-2+IL-4 in the presence or absence of 1,25D3 (100 nM, or 1 μM). Representative results of intracellular staining of IFN-γ and IL-13 expression in CD8⁺ T cells with or without SIINFEKL (SEQ ID NO:1) (T cell receptor, TCR) restimulation.

FIGS. 2A-2F show 1,25D3 treatment of CD8⁺ T cells alters gene expression of transcription factors and the functional activity of CYP11A1. Gene expression of Gata3 (FIG. 2A), Tbx21 (FIG. 2B), Cyp11a1 (FIG. 2C), and Vdr (FIG. 2D) was measured by quantitative PCR (qPCR) in CD8⁺ T cells in IL-2 or IL-2+IL-4 in the presence of absence of 100 nM or 1 μM 1,25D3. Results (relative fold change+SEM) are from at least three independent experiments. CYP11A1 protein levels (mean+SEM) detected by immunoblot analyses and densitometry of autoradiographs in CD8⁺ T cells differentiated in IL-2 or IL-2+IL-4 in the presence of absence of 100 nM or 1 μM 1,25D3 (FIG. 2E). Pregnenolone levels (mean+SEM) determined by ELISA in supernatants from CD8⁺ T cells differentiated in IL-2 or IL-2+IL-4 in the presence or absence of 100 nM or 1 μM 1,25D3 (FIG. 2F). Results are from six independent experiments. Linear mixed models were employed; pairwise comparisons were performed using t-test derived from these models. *p<0.05, **p<0.01, ***p<0.001 compared with the IL-2 group, ###p<0.001 compared to the IL-2+IL-4 group, these p-values remained significant after correction for multiple comparisons (Benjamini-Hochberg (Moffatt, M. F., et al. Genetic variants regulating ORMDL3 expression contribute to the risk of childhood asthma. Nature 448, 470-473 (2007)); p-values that did not reach the threshold p-value after adjustment for multiple comparisons are shown numerically.

FIGS. 3A-3B show 1,25D3 mediated VDR recruitment to the Cyp11a1 promoter region is altered in CD8⁺ T cells differentiated in IL-2 or IL-2+IL-4 in the presence or absence of 100 nM or 1 μM 1,25D3. Localization of VDR-binding sites and qPCR primers in the Cyp11a1 promoter region (FIG. 3A). qPCR was performed using five Cyp11a1 promoter specific primers covering seven VDR-binding sites. Data were analyzed via the percent input methodology: (2̂(CT of total input−CT of specific IP))×100 and relative percent input ratios using CD8⁺ T cells stimulated with IL-2 as baseline. Data (relative fold change+SEM) are from three independent experiments. Linear mixed models were employed; pairwise comparisons were performed using t-tests derived from these models. *p<0.05, **p<0.01, ***p<0.001 compared to the IL-2 group, these p-values remained significant after correction for multiple comparisons; (Benjamini-Hochberg (Moffatt, M. F., et al. Genetic variants regulating ORMDL3 expression contribute to the risk of childhood asthma. Nature 448, 470-473 (2007)). No statistical significance was detected compared to the IL-2+IL-4 group.

FIGS. 4A-4B show the adoptive transfer of 1,25D3-treated CD8⁺ T cells into CD8-deficient recipients fails to induce AHR. Recipient mice were sensitized (two intraperitoneal, i.p.) and challenged (4 nebulizations, Neb) using secondary allergen challenge model and received no cells, CD8⁺ T cells differentiated in IL-2 alone (CD8) or in the presence of 100 nM (CD8+100 nM 1,25D3) or 1 μM 1,25D3 (CD8+1 μM 1,25D3). FIG. 4A: changes in airway resistance (RL) were measured in response to increasing concentrations of methacholine. FIG. 4B: Cell composition in BAL fluid. Data (mean+SEM) are from two to three experiments with three to four mice per experiment. *p<0.05, **p<0.01, ***p<0.001; compared to sensitized and challenged CD8-deficient recipients which received no cells. General linear models were employed; pairwise comparisons were performed using t=test derived from these models. #p<0.05, ##p<0.01, ###p<0.001 compared with sensitized and challenged CD8-deficient recipients which received CD8⁺ T cells differentiated in IL-2 alone, these p-values remained significant after correction for multiple comparisons; (Benjamini-Hochberg (Moffatt, M. F., et al. Genetic variants regulating ORMDL3 expression contribute to the risk of childhood asthma. Nature 448, 470-473 (2007)). p-values that did not reach the threshold p-value after adjustment for multiple comparisons are shown numerically.

FIGS. 5A-5C show adoptive transfer of 1,25D3-treated CD8⁺ T cells into CD8-deficient recipients decreased cytokine levels in the BAL. Cytokine levels in BAL fluid (mean+SEM): (FIG. 5A) IL-4, (FIG. 5B) IL-5, and (FIG. 5) IL-13. *p<0.05, **p<0.01; compared to sensitized and challenged CD8-deficient recipients that received no cells. Data (mean+SEM) are from two to three experiments with three to four mice per experiment. General linear models were employed; pairwise comparisons were performed using t-tests derived from these models. #p<0.05, ##p<0.01, ###p<0.001 compared with sensitized and challenged CD8-deficient recipients which received CD8⁺ T cells differentiated in IL-2 alone, p-values remained significant after correction for multiple comparisons (Benjamini-Hochberg (Moffatt, M. F., et al. Genetic variants regulating ORMDL3 expression contribute to the risk of childhood asthma. Nature 448, 470-473 (2007)).

FIGS. 6A-6E show adoptive transfer of 1,25D3-treated CD8⁺ T cells into CD8-deficient recipients prevents goblet cell metaplasia. Representative photomicrographs of lung histology (original magnification ×3200) (FIG. 6A, FIG. 6B, FIG. 6C, FIG. 6D). Quantitative analysis (FIG. 6E) of PAS-positive goblet cells was as determined in cross-sectional areas of the airway wall. Data (mean+SEM) are from two experiments with three mice per experiment. Linear mixed models were employed; pairwise comparisons were performed using t-test derived from these models. **p<0.01 these p-values remained significant after correction for multiple comparisons; p-values that did not reach the threshold p-value after adjustment for multiple comparisons (Benjamini-Hochberg (Moffatt, M. F., et al. Genetic variants regulating ORMDL3 expression contribute to the risk of childhood asthma. Nature 448, 470-473 (2007)) are shown numerically.

FIG. 7 shows 1,25D3-mediated VDR recruitment to the Cyp11a1 promoter region is altered in CD8⁺ T cells after adoptive transfer into sensitized and challenged CD8-deficient mice. CD8⁺ T cells were differentiated in vitro in IL-2 in the presence or absence of 1 μM 1,25D3. CD8⁺ T cells from the lungs were recovered after adoptive transfer into sensitized and challenged CD8-deficient mice (n=3 mice/group). qPCR was performed using five Cyp11a1 promoter specific primers covering seven VDR binding sites. Data were analyzed via the percent input methodology: (2̂ (CT of total input−CT of specific IP))×100.

FIG. 8 shows that the cell viability of CD8⁺ T cells differentiated in IL-2 or IL-2+IL-4 is not altered by 1,25D3 (100 nM or 1 μM). Total cell numbers were counted on days 0 and 4 during CD8⁺ T cells differentiation in IL-2 or IL-2+IL-4 in the presence or absence of 100 nM or 1 μM 1,25D3. Data (mean±SEM) are from five experiments. **p<0.01 compared to the IL-2 group on day 4.

FIGS. 9A-9B show the presence of 1,25D3 only during the antigen re-stimulation phase does not alter IFN-γ and IL-13 production in CD8⁺ T cells differentiated in IL-2 or IL-2+IL-4.

FIG. 9A shows the representative results of intracellular staining and FIG. 9B shows the data (mean+/−SEM) showing percent positive cells (four independent experiments) for IFN-γ and IL-13 expression in CD8⁺ T cells differentiated in IL-2 or IL-2+IL-4 with SIINFEKL (SEQ ID NO:1) (T cell receptor, TCR) restimulation. 1,25D3 (100 nM or 1 μM) was added for 4 hours only during the antigen restimulation phase.

FIG. 10 shows the 1,25D3 treatment of CD8+T cells alters CYP11A1 protein levels. Representative results of a CYP11A1 western blot of CD8+ T cells differentiated in IL-2 or IL-2+IL4 in the presence or absence of 100 nM or 1 uM 1,25D3. * calculated molecular weight (MW) of CYP11A1 (13363-1-AP, Proteintech, Chicago, Ill.): 60 kDA, observed MW: 49 kDA.

FIG. 11 shows the genomic structure of Cyp11a1 and localization of VDR binding sites in the Cyp11a1 promoter region. Exons are displayed in black and smaller grey boxes represent 5′ and 3′ untranslated regions (UTR). VDR binding sites in the Cyp11a1 promoter and respective Cyp11a1 -specific primers are depicted relative to the location of the CYP11A1 coding region.

FIGS. 12A-12E show 1,25D3-mediated VDR recruitment to the Cyp11a1 promoter region is altered in CD8⁺ T cells differentiated in IL-2 or IL-2+IL-4 in the presence or absence of 100 nM or 1 μM 1,25D3. A representative experiment of the percent input immunoprecipitated by the VDR and the negative control IgG antibody. qPCR was performed using 5 Cyp11a1 promoter-specific primers [FIG. 12A, VDR 6+7; FIG. 12B, VDR 4+5; FIG. 12C, VDR 3; FIG. 12D, VDR 2; and FIG. 12E, VDR 1] covering seven VDR binding sites. Data were analyzed via the percent input methodology: (2̂(CT of total input−CT of specific IP))×100.

FIG. 13 shows the allele-specific effects of rs8076131 and rs4065275 on CYP11A1 promoter activity as tested in a luciferase reporter system in HEK293 cells using pGL4 plasmids (Promega, Madison, USA) containing different combinations of the two SNPs in the CYP11A1 promoter region (pGZ14, pGZ15, pGZ17, pGZ16). The pRL-TK Renilla luciferase reporter plasmid (Promega, Madison, USA) was co-transfected for normalization of transfection efficiency and cell viability. Cells were in medium alone or stimulated (1 μM Vitamin D3 (1,25D3), 20 ngml IL4, 1 μM Vitamin D3 (1,25D3)+20 ngml IL4, 100 nM dexamethasone, or 50 ng/ml PMA/1 μM ionomycin) 3 h after transfection and dual luciferase reporter assays were performed 21 h later.

### DETAILED DESCRIPTION

The invention generally relates to methods for the prevention and/or treatment of an allergic disease or condition in subjects who have or are at risk of developing an allergic disease or condition, as well as, methods of preventing an acute allergic response in subjects in need thereof. The invention includes administration of a therapeutically effective amount of 1,25-dihydroxy vitamin D3 (1,25D3). In one aspect, the methods further comprise administering a steroidogenic pathway inhibitor. The invention includes the use of a composition comprising 1,25D3 as well as the composition itself. The invention also includes kits that contain one 1,25D3 with or without one or more steroidogenic pathway inhibitors.

Effector CD8⁺ T cells convert from IFN-γ⁺ to IL-13⁺ cells in the presence of IL-4. The inventors have determined the surprising result that addition of 1,25D3 (the active form of Vitamin D3) during the differentiation of CD8⁺ T cells, prevented IL-4-induced conversion to IL-13-producers. Unlike recipients of non-treated cells, transfer of 1,25D3-treated CD8⁺ T cells into sensitized and challenged CD8⁺ -deficient recipients failed to restore development of lung allergic responses. 1,25D3 altered vitamin D receptor (VDR) recruitment to the Cyp11a1 promoter region in vitro and in vivo in the presence of IL-4. As a result, protein levels and enzymatic activity of CYP11A1, a steroidogenic enzyme which regulates CD8⁺ T-cell conversion, were decreased. Thus, an epistatic effect between CYP11A1 and VDR polymorphisms appears to contribute to the genetic predisposition of childhood asthma. These findings identify a novel role for 1,25D3 in the molecular programming of CD8⁺ T-cell conversion to an IL-13-secreting phenotype through regulation of steroidogenesis, governing asthma susceptibility thus providing for prevention means and/or therapeutic means for preventing and/or treating allergic disease and/or acute allergic responses.

According to the present invention, allergic diseases and/or conditions, include but are not limited to pulmonary conditions such as allergic lung disease, allergic rhinitis, asthma, steroid refractive asthma, airway hyperresponsiveness, allergen-induced airway hyperresponsiveness and hay fever as well as other allergic conditions including but not limited to a food allergy, allergen-induced inflammation, eosinophilic esophagitis, chronic urticaria, atopic dermatitis, occupational allergy, allergic conjunctivitis, airborne allergic sensitivities, stinging insect allergy, hypersensitivity pneumonitis, eosinophilic lung diseases, inflammatory bowel disease, ulcerative colitis, Crohn's disease and drug allergies. Symptoms of the allergies, including but not limited to diarrhea and intestinal inflammation as well as asthma and airway hyperresponsiveness, is apparently or obviously, directly or indirectly triggered by an allergen to which a subject has previously been sensitized. In one aspect, the allergic disease or condition can be caused by one or more proteinaceous allergens. Sensitization to an allergen refers to being previously exposed one or more times to an allergen such that an immune response is developed against the allergen. Responses associated with an allergic reaction, including but not limited to histamine release, edema, vasodilatation, bronchial constriction, airway inflammation, airway hyperresponsiveness, asthma, allergic rhinitis (hay fever), nasal congestion, sneezing, running nose, skin rash, diarrhea including acute allergic diarrhea and intestinal inflammation), typically do not occur when a naive subject is exposed to the allergen for the first time, but once a cellular and humoral immune response is produced against the allergen, the subject is “sensitized” to the allergen. Allergic reactions then occur when the sensitized individual is re-exposed to the same allergen (e.g., an allergen challenge). Once a subject is sensitized to an allergen, the allergic reactions can become worse with each subsequent exposure to the allergen, because each re-exposure not only produces allergic symptoms, but further increases the level of antibody produced against the allergen and the level of T cell response against the allergen.

In one aspect of the invention, exposure to an allergen results in an acute allergic reaction, thus the invention provides for preventing an acute allergic reaction in a subject by administering a therapeutically effective amount of 1,25D3. An acute allergic reaction occurs when the body reacts to a certain substance (allergens) by releasing histamines. Symptoms of an acute allergic reaction or response can include sneezing, a runny nose, rashes, swollen skin, hives, and difficulty breathing. The symptoms often commence within minutes after exposure to the allergen. An acute allergic reaction can occur when the subject exposed to common allergens such as pet dander, shellfish, peanuts, wheat, and dairy. A sudden, severe allergic reaction can be fatal for subject having the acute allergic reaction. Some subjects are genetically predisposed to having acute allergy reactions. Early exposure to certain allergy-causing substances can cause subjects to develop severe, lifelong allergies against it. The genetic component is clear, however, as allergies tend to run in families. The body thinks that a harmless substance is a danger, so it produces reactions designed to repel and destroy the offender. If left untreated, an acute allergic reaction can result in death.

According to the present invention, inflammation is characterized by the release of inflammatory mediators (e.g., cytokines or chemokines) which recruit cells involved in inflammation to a tissue. A condition or disease associated with allergic inflammation is a condition or disease in which the elicitation of one type of immune response (e.g., a Th2-type immune response) against a sensitizing agent, such as an allergen, can result in the release of inflammatory mediators that recruit cells involved in inflammation in a subject, the presence of which can lead to tissue damage and sometimes death. A Th2-type immune response is characterized in part by the release of cytokines which include IL-4, IL-5, and IL-13. A TH17-type response is characterized by the release of IL-17. The present invention is particularly useful for treating allergen-induced food allergies (such as peanut allergies) and airway hyperresponsiveness and airway inflammation, including, allergen-induced asthma and rhinitis.

Accordingly, various embodiments of the present invention include treating a subject that has been sensitized to an allergen and has been or is at risk of becoming exposed to the allergen.

In other embodiments, the present invention includes preventing an allergic disease or condition in a subject at risk of becoming exposed to the allergen. In still another aspect, the present invention provides for preventing an acute allergic response in a subject. Such allergens can be related to a food, a plant, a gas, a pathogen, a metal, a glue or a drug. Examples of food allergens include but are not limited to groundnuts such as peanuts; nuts from trees including Brazilian nuts, hazelnuts, almonds, walnuts; fruit, milk, eggs, fish, shellfish, wheat, or gluten. Examples of plant allergens include but are not limited to pollen, trees, grass, weeds, ragweed, poison Oak or poison ivy. Examples of gas allergens include but are not limited to environmental tobacco smoke, and carbon monoxide. Examples of pathogen allergens include but are not limited to mold, viruses or bacteria. Examples of metal allergens include but are not limited to lead, nickel, chromate, or cobalt. Examples of drug allergens include but are not limited to penicillin, sulfur, or aspirin. Additional allergens include but are not limited to latex, dust mites, pet dander (skin flakes), droppings from cockroaches, rodents and other pests or insects.

According to the present invention, “airway hyperresponsiveness” or “AHR” refers to an abnormality of the airways that allows them to narrow too easily and/or too much in response to a stimulus capable of inducing airflow limitation. AHR can be a functional alteration of the respiratory system resulting from inflammation in the airways or airway remodeling (e.g., such as by collagen deposition). Airflow limitation refers to narrowing of airways that can be irreversible or reversible. Airflow limitation or airway hyperresponsiveness can be caused by collagen deposition, bronchospasm, airway smooth muscle hypertrophy, airway smooth muscle contraction, mucous secretion, cellular deposits, epithelial destruction, alteration to epithelial permeability, alterations to smooth muscle function or sensitivity, abnormalities of the lung parenchyma and infiltrative diseases in and around the airways. Many of these causative factors can be associated with inflammation. AHR can be triggered in a patient with a condition associated with the above causative factors by exposure to a provoking agent or stimulus. Such stimuli include, but are not limited to, an allergen.

According to the present invention, treatment of a subject having an allergic disease and/or condition can commence as soon as it is recognized (i.e., immediately) by the subject or by a clinician, that the subject has been exposed or is about to be exposed to an allergen. Additionally, preventing an allergic disease or condition can commence prior to the subject being exposed to an allergen. Treating the subject and/or preventing an allergic disease or condition and/or preventing an acute allergic response in the subject, comprises administering a therapeutically effective amount of a composition comprising 1,25D3. In one aspect, the subject is further administered, a therapeutic effective amount of one or more steroidogenic pathway inhibitors either concurrently, prior to or following administration of the 1,25D. The steroidogenic pathway inhibitors include but are not limited to a small molecule inhibitor, an antibody, a chemical entity, a nucleotide, a peptide, a protein, an antisense molecule, and siRNA molecule, and shRNA molecule that inhibits one or more proteins, and/or protein-by-products, enzymes, and/or receptors of the steroidogenic pathway. Inhibiting a component of the steroidogenic pathway includes both direct inhibition of the components as well as inhibition of the expression of the one or more components of the pathway. Inhibition of one or more components of the steroidogenic pathway can be by any mechanism, including, without limitations, decreasing activity of one or more components, increasing inhibition of one or more of the components, degradation of one or more of components, a reduction or elimination of expression of one or more components and combinations thereof. Binding to one or more component to prevent its wild-type enzymatic activity for example, including competitive and noncompetitive inhibition, inhibiting transcription, and regulating expression can also inhibit the component. These inhibitors can also reduce expression of CD4⁺ and CD8⁺ T cell proliferation and have the ability to suppress Th2 differentiation and/or Th17 differentiation. In still one aspect, the steroidogenic pathway inhibitor interacts with a regulator of a component of the steroidogenic pathway including but not limited to Cyp11A1 mRNA expression or Cyp11A1 protein expression. In one aspect, the regulator is an inhibitor of the steroidogenic pathway, including but not limited to an antibody, an antisense molecule, an siRNA molecule, an shRNA molecule, a receptor antagonist, a chemical entity, a nucleotide, a peptide and a protein. In one aspect, the steroidogenic pathway inhibitor inhibits one or more enzymes, receptors, or protein by-products of the steroidogenic pathway. In a preferred embodiment, the steroidogenic pathway inhibitor inhibits Cyp11A. This inhibitor can be aminoglutetiamide, a Cyp11A siRNA molecule or a Cyp11A shRNA molecule. In other aspect, the steroidogenic pathway inhibitor inhibits 3βHSD and can be trilostane. In still another aspect, the steroidogenic pathway inhibitor inhibits Cyp11β1 (cytochrome P450 family 11 subfamily β polypeptide 1) and can be metyrapone.

In accordance with the present invention, acceptable protocols to administer a composition of the present invention including the route of administration and the effective amount of a composition to be administered to a subject can be determined by those skilled in the art. The composition of the present invention can be administered in vivo or ex vivo. Suitable in vivo routes of administration can include, but are not limited to, aerosol, oral, nasal, inhaled, topical, intratracheal, transdermal, rectal, or parenteral routes. Preferred parenteral routes can include, but are not limited to, subcutaneous, intradermal, intravenous, intramuscular, or intraperitoneal routes.

The invention also includes kits that contain 1,25D3. In a further aspect, the kit further contains one or more steroidogenic pathway inhibitors. In addition, according to the present invention, the composition as well as the kits of the present invention, can comprise a pharmaceutically acceptable excipient. According to the present invention, the composition, may be administered with a pharmaceutically acceptable carrier, which includes pharmaceutically acceptable excipients and/or delivery vehicles, for delivering the agent to a subject (e.g., a liposome delivery vehicle). As used herein, a pharmaceutically acceptable carrier refers to any substance suitable for delivering a therapeutic composition useful in the method of the present invention to a suitable in vivo or ex vivo site. Suitable excipients of the present invention include excipients or formularies that transport or help transport, but do not specifically target a nucleic acid molecule to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters, glycols and combinations thereof. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Compositions of the present invention can be sterilized by conventional methods and/or lyophilized.

According to the methods of the present invention, the subject can be any animal subject, and particularly, in any vertebrate mammal, including, but not limited to, primates, rodents, livestock or domestic pets. Preferred mammals for the methods of the present invention include humans. In a preferred aspect, the subject has been determined to have steroid-insensitive asthma and/or steroid-insensitive CD8+ cells.

Although cluster designations have identified some of the clinical features in asthmatics, actual definition of underlying pathobiology has lagged behind, thus limiting targeted therapy. To date, only one endotype has been well-characterized, a T_{H}2-high-signature associated with CD4⁺ T cells, IL-13, and steroid-responsiveness. There has been accumulating evidence for the role of CD8⁺ T cells in asthma, especially in steroid-refractory asthma (Hamelmann, E., et al. Requirement for CD8⁺ T cells in the development of airway hyperresponsiveness in a murine model of airway sensitization. J. Exp. Med. 183, 1719-1729 (1996); Isogai, S., et al. CD8⁺ alphabeta T cells can mediate late airway responses and airway eosinophilia in rats. J. Allergy Clin. Immunol. 114, 1345-1352 (2004); Jia, Y., et al. Steroidogenic enzyme CYP11A1 regulates type 2 CD8⁺ T cell skewing in allergic lung disease. Proc. Natl. Acad. Sci. USA 110, 8152-8157 (2013); Miyahara, N., et al. Leukotriene B4 release from mast cells in IgE-mediated airway hyperresponsiveness and inflammation. Am. J. Respir. Cell Mol. Biol. 40, 672-682 (2009); Miyahara, N., et al. Effector CD8⁺ T cells mediate inflammation and airway hyper-responsiveness. Nat. Med. 10, 865-869 (2004); Miyahara, N., et al. Contribution of antigen-primed CD8⁺ T cells to the development of airway hyperresponsiveness and inflammation is associated with IL-13. J. Immunol. 172, 2549-2558 (2004); Miyahara, N., et al. Requirement for leukotriene B4 receptor 1 in allergen-induced airway hyperresponsiveness. Am. J. Respir. Crit. Care Med. 172, 161-167 (2005); Taube, C., et al. The leukotriene B4 receptor (BLT1) is required for effector CD8⁺ T cell-mediated, mast cell-dependent airway hyperresponsiveness. J. Immunol. 176, 3157-3164 (2006); Li, L. B., Leung, D. Y., Strand, M. J. & Goleva, E. ATF2 impairs glucocorticoid receptor-mediated transactivation in human CD8⁺ T cells. Blood 110, 1570-1577 (2007); Gelfand, E. W. & Dakhama, A. CD8⁺ T lymphocytes and leukotriene B4: novel internnovel interactions in the persistence and progression of asthma. J. Allergy Clin. Immunol. 117, 577-582 (2006); van Rensen, E. L., et al. Bronchial CD8 cell infiltrate and lung function decline in asthma. Am. J. Respir. Crit. Care Med. 172, 837-841 (2005); Chung, E. H., et al. Leukotriene B4 receptor 1 is differentially expressed on peripheral T cells of steroid-sensitive and -resistant asthmatics. Ann. Allergy Asthma Immunol. 112, 211-216 e211 (2014)). The inventors have shown in vitro and in vivo that in the presence of IL-4, mouse CD8⁺ T cells can convert from IFN-γ-producers to a significant source of IL-13 (Jia, Y., et al. Steroidogenic enzyme CYP11A1 regulates type 2 CD8⁺ T cell skewing in allergic lung disease. Proc. Natl. Acad. Sci. USA 110, 8152-8157 (2013); Jia, Y., et al. Stepwise epigenetic and phenotypic alterations poise CD8⁺ T cells to mediate airway hyperresponsiveness and inflammation. J. Immunol. 190, 4056-4065 (2013)). This conversion was associated with changes in lineage-specific transcription factor signatures and histone modifications at key loci. In the terminal stage of differentiation to IL-13 production, the enzymatic activation of CYP11A1 was shown to play an essential role ((Jia, Y., et al. Steroidogenic enzyme CYP11A1 regulates type 2 CD8⁺ T cell skewing in allergic lung disease. Proc. Natl. Acad. Sci. USA 110, 8152-8157 (2013); Jia, Y., et al. Stepwise epigenetic and phenotypic alterations poise CD8⁺ T cells to mediate airway hyperresponsiveness and inflammation. J. Immunol. 190, 4056-4065 (2013)). Since vitamin D deficiency has been implicated in refractory asthma, although without a defined molecular mechanism, the inventors determined if 1,25D3 modulated the conversion of IL-13⁺CD8⁺ T cells, a pathway shown to play a role in asthma ((Jia, Y., et al. Steroidogenic enzyme CYP11A1 regulates type 2 CD8⁺ T cell skewing in allergic lung disease. Proc. Natl. Acad. Sci. USA 110, 8152-8157 (2013); Jia, Y., et al. Stepwise epigenetic and phenotypic alterations poise CD8⁺ T cells to mediate airway hyperresponsiveness and inflammation. J. Immunol. 190, 4056-4065 (2013)). The inventors now show the surprising result that differentiation of CD8⁺ T cells in the presence of 1,25D3 prevented the IL-4-mediated skewing of CD8⁺ T cells to IL-13-producing Tc2 cells. As a consequence, adoptively-transferred CD8⁺ T cells, differentiated in the presence of 1,25D3, failed to induce lung allergic responses in sensitized and challenged CD8-deficient recipients. This failure links 1,25D3 induced changes in the binding of the VDR transcription factor to the Cyp11a1 promoter region. In humans, the data supported an epistatic effect between CYP11A1 and VDR polymorphisms, diminishing the risk to develop childhood asthma.

Vitamin D has previously been shown to play a critical role in modulating effects of CD4⁺ T cell subsets (Chambers, E. S., et al. Serum 25-dihydroxyvitamin D levels correlate with CD4⁺Foxp3⁺ T-cell numbers in moderate/severe asthma. J. Allergy Clin. Immunol. 130, 542-544 (2012); Matheu, V., Back, O., Mondoc, E. & Issazadeh-Navikas, S. Dual effects of /T_{H}2 cytokine expression: enhancing IgE production and decreasing airway eosinophilia in murine allergic airway disease. J. Allergy Clin. Immunol. 112, 585-592 (2003); Nanzer, A. M., et al. Enhanced production of IL-17A in patients with severe asthma is inhibited by 1 alpha,25-dihydroxyvitamin D3 in a glucocorticoid-independent fashion. J. Allergy Clin. Immunol. 132, 297-304 e293 (2013)) and even enhanced the therapeutic response(s) to corticosteroids (Xystrakis, E., et al. Reversing the defective induction of IL-10-secreting regulatory T cells in glucocorticoid-resistant asthma patients. J. Clin. Invest. 116, 146-155 (2006)). However, little mechanistic data are available on the impact of 1,25D3 on CD8⁺ T cells. When 1,25D3 was added during CD8⁺ T cell differentiation, the IFN-γ-producing capacity of Tc1 cells differentiated in IL-2 alone was not affected. However, the cytokine profile in CD8⁺ T cells differentiated in IL-2+IL-4 was significantly modified, characterized by increased IFN-γ and decreased IL-13 production. These findings by the inventors indicate that the activity of 1,25D3 was dependent on the exposure to IL-4 during CD8⁺ T cell skewing, which can mediate the epigenetic poising of CD8⁺ T cells at loci sensitive to VDR activity. Thus, it is possible that fully differentiated CD8⁺ Tc2 cells are insensitive to 1,25D3 due to previous changes in chromatin structure at critical loci, now insensitive to VDR.

In light of the immunomodulatory activities associated with vitamin D (Cantorna, M. T., Zhu, Y., Froicu, M. & Wittke, A. Vitamin D status, 1,25-dihydroxyvitamin D3, and the immune system. Am. J Clin. Nutr. 80, 1717S-1720S (2004); Cantorna, M. T. & Mahon, B. D. D-hormone and the immune system. J Rheumatol Suppl 76, 11-20 (2005); Hayes, C. E., Nashold, F. E., Spach, K. M. & Pedersen, L. B. The immunological functions of the Vitamin D endocrine system. Cell Mol. Biol. 49, 277-300 (2003)), there has been increased interest in determining its role in asthma. However, results of clinical trials using vitamin D as a therapeutic supplement (Gupta, A., et al. Relationship between serum Vitamin D, disease severity, and airway remodeling in children with asthma. Am. J Respir. Crit. Care Med. 184, 1342-1349 (2011); Brehm, J. M., et al. Serum Vitamin D levels and severe asthma exacerbations in the Childhood Asthma Management Program study. J. Allergy Clin. Immunol. 126, 52-58 e55 (2010); Goleva, E., Searing, D. A., Jackson, L. P., Richers, B. N. & Leung, D. Y. Steroid requirements and immune associations with Vitamin D are stronger in children than adults with asthma. J. Allergy Clin. Immunol. 129, 1243-1251 (2012); Korn, S., Hubner, M., Jung, M., Blettner, M. & Buhl, R. Severe and uncontrolled adult asthma is associated with Vitamin D insufficiency and deficiency. Respir. Res. 14, 25 (2013); Searing, D. A., et al. Decreased serum Vitamin D levels in children with asthma are associated with increased corticosteroid use. J. Allergy Clin. Immunol. 125, 995-1000 (2010); Castro, M., et al. Effect of Vitamin D3 on asthma treatment failures in adults with symptomatic asthma and lower Vitamin D levels: the VIDA randomized clinical trial. J. Am. Med. Association 311, 2083-2091 (2014); Carraro, S., et al. Asthma severity in childhood and metabolomic profiling of breath condensate. Allergy 68, 110-117 (2013); Gergen, P. J., et al. Lack of a relation between serum 25-hydroxyvitamin D concentrations and asthma in adolescents. Am. J. Clin. Nutr. 97, 1228-1234 (2013); Tolppanen, A. M., et al. Prospective association of 25-hydroxyvitamin D3 and D2 with childhood lung function, asthma, wheezing, and flexural dermatitis. Epidemiology 24, 310-319 (2013)) or during pregnancy to attenuate development of atopic disease in progeny (Chawes, B. L., et al. Cord blood 25(OH)-vitamin D deficiency and childhood asthma, allergy and eczema: the COPSAC2000 birth cohort study. PLoS One 9, e99856 (2014); Goldring, S. T., et al. Prenatal Vitamin D supplementation and child respiratory health: a randomised controlled trial. PLoS One 8, e66627 (2013); Jones, A. P., Palmer, D., Zhang, G. & Prescott, S. L. Cord blood 25-hydroxyvitamin D3 and allergic disease during infancy. Pediatrics 130, e1128-1135 (2012); Nwaru, B. I., et al. Maternal diet during pregnancy and allergic sensitization in the offspring by 5 yrs of age: a prospective cohort study. Pediatr. Allergy Immunol. 21, 29-37 (2010); Rosenwasser, L. J. & Borish, L. Genetics of atopy and asthma: the rationale behind promoter-based candidate gene studies (IL-4 and IL-10). Am. J. Respir. Crit. Care Med. 156, S152-155 (1997); Weisse, K., et al. Maternal and newborn Vitamin D status and its impact on food allergy development in the German LINA cohort study. Allergy 68, 220-228 (2013)) have been conflicting. The inventors' results provided herein, indicate that 1,25D3 supplementation exhibits preventive activities rather than increase the therapeutic potential during an asthma exacerbation as the molecular mechanisms implicated in responsive cells at that stage may have already been epigenetically poised; however, by modifying transcription of key pro-allergic transcription factor expression an early stage may attenuate disease progression. Further, 1,25D3 as an interventional strategy may be restricted to a well-defined subpopulation of steroid-refractory asthmatics with increased numbers of CD8⁺ IL-13⁺ T cells (Tc2 cells).

As further provided in the examples below, during differentiation of CD8⁺ T cells in the presence of IL-4 and 1,25D3, lineage-specific markers (e.g., Gata3 and Tbx21) were only marginally altered whereas CYP11A1 gene and protein expression were significantly affected in a dose-dependent manner. Changes at a transcriptomic level appeared weaker, likely the result of analysis on day 4 of the CD8⁺ T cell differentiation protocol. Importantly, the enzymatic activity of CYP11A1, as measured by generation of pregnenolone was markedly reduced. Together with T-cell receptor engagement (TCR engagement), the relevance of this essential factor in the conversion of CD8⁺ T cells to IL-13-producers was augmented. 1,25D3 was only effective when included during CD8⁺ differentiation and was without effect when added during the terminal differentiation stage.

Adoptive transfer of 1,25D3 treated CD8⁺ T cells into sensitized and challenged CD8-deficient mice failed to induce lung allergic responses. Similar inhibitory effects on conversion were observed following inhibition of the enzymatic activity of CYP11A1 (with aminoglutethimide (AMG)) or silencing of the gene (Jia, Y., et al. Steroidogenic enzyme CYP11A1 regulates type 2 CD8⁺ T cell skewing in allergic lung disease. Proc. Natl. Acad. Sci. USA 110, 8152-8157 (2013)) as both were associated with the failure to restore lung allergic responses in sensitized and challenged CD8-deficient mice. These results indicate that 1,25D3, similar to AMG, acts upstream of CYP11A1. The effects on mouse CD8⁺ T cells required 100 nM to 1 mM concentrations of 1,25D3, concentration (500 nM calcitriol) previously demonstrated in vitro to be effective in CD4⁺ T cells from steroid-resistant asthmatics (Campbell, F. C., Xu, H., El-Tanani, M., Crowe, P. & Bingham, V. The yin and yang of vitamin D receptor (VDR) signaling in neoplastic progression: operational networks and tissue-specific growth control. Biochem. Pharmacol. 79, 1-9 (2010)). These results indicate that in vitro, higher concentrations of 1,25D3 are needed to mimic potential in vivo effects.

Little is known about molecular mechanisms mediated by 1,25D3 in CD8⁺ T cells, specifically in the conversion to IL-13 production and contributions to asthma pathogenesis. The active form of vitamin D primarily transmits a signal through the transcription factor VDR, a member of the nuclear hormone receptor family (Campbell, F. C., Xu, H., El-Tanani, M., Crowe, P. & Bingham, V. The yin and yang of vitamin D receptor (VDR) signaling in neoplastic progression: operational networks and tissue-specific growth control. Biochem. Pharmacol. 79, 1-9 (2010)). By performing ChIP experiments with a VDR-specific antibody, the inventors show that VDR acts as a transcriptional repressor of CYP11A1; in IL-4-activated CD8⁺ T cells, the recruitment of VDR to Cyp11a1 was decreased leading to higher CYP11A1 expression, whereas in CD8⁺ cells cultured in IL-2 alone, more VDR binding paralleled by lower CYP11A1 expression was detected. A similar effect on IL-12B expression has previously been reported in lipopolysaccharide (LPS)-treated human monocytes (THP-1) (Gynther, P., et al. Mechanism of 1alpha,25-dihydroxyvitamin D(3)-dependent repression of interleukin-12B. Biochim. Biophy.s Acta. 1813, 810-818 (2011). Vitamin D induced alterations in the recruitment of VDR/RXR, the co-repressor NCOR2/SMRT, and histone deacetylase 3 paralleled by decreased histone 4 acetylation (H4ac), and increased histone 3 trimethylation (H3K27me3), potentially causing the downregulation of IL-12B. In the CD8⁺ T cell differentiation model disclosed herein, 1,25D3-induced recruitment of VDR to the Cyp11a1 promoter region resulted in lower CYP11A1 enzymatic activity, which in turn, is associated with attenuation of the IL-4-driven conversion of CD8⁺ T cells to IL-13 production. Examination of adoptively-transferred CD8⁺ T cells recovered from the lungs of sensitized and challenged recipient mice recapitulated these in vitro findings, as shown by the increased recruitment of VDR to the Cyp11a1 promoter region in recovered cells from recipients of 1,25D3-treated cells. These in vitro and in vivo results indicate that CYP11A1 is an important regulator of the conversion of CD8⁺ T cells and accounts for the failure of transferred 1,25D3-treated cells to restore lung allergic responses.

To determine if these findings in mice are relevant to asthma, the inventors determined if CYP11A1 is involved in the susceptibility of asthma in childhood. Three CYP11A1 tagging SNPs decreased the risk for asthma in a case-control population. Even though, the effect did not reach genome-wide statistical significance, the inventors' previous findings in experimental asthma supported the relevance of CYP11A1. Most of the CYP11A1 polymorphisms covered by the asthma-associated tagging SNPs were located in regulatory regions of CYP11A1, indicating a potential allele-specific function. Depending on the genotype of rs8039957, located in the CYP11A1 promoter region, a change in VDR binding was predicted. Hence, a similar regulatory mechanism of CYP11A1 through VDR as observed in mice may be relevant to human disease. An interaction between asthma-associated SNPs in CYP11A1 and VDR supported the relevance of CYP11A1 and VDR predisposing to childhood asthma. However, genetic variants in VDR have been studied extensively in the context of asthma with conflicting results (Bosse, Y., et al. Asthma and genes encoding components of the Vitamin D pathway. Respir. Res. 10, 98 (2009); Michel, S., et al. Unifying candidate gene and GWAS approaches in asthma. PloS One 5, e13894 (2010); Maalmi, H., et al. Association of vitamin D receptor gene polymorphisms with susceptibility to asthma in Tunisian children: A case control study. Hum. Immunol. 74, 234-240 (2013); Raby, B. A., et al. Association of vitamin D receptor gene polymorphisms with childhood and adult asthma. Am. J. Respir. Crit. Care Med. 170, 1057-1065 (2004); Vollmert, C., et al. Single nucleotide polymorphism screening and association analysis--exclusion of integrin beta 7 and vitamin D receptor (chromosome 12q) as candidate genes for asthma. Clin. Exp. Allergy 34, 1841-1850 (2004)).

As provided for in the examples below, the data establish for the first time a mechanistic role for vitamin D (1,25D3) in regulating the IL-4-mediated conversion of CD8⁺ T cells to IL-13-producing pathogenic effector cells. This positions a novel role for 1,25D3 as a critical regulator of the development of lung allergic responses through a unique VDR-CYP11A1-IL-13 pathway in steroid-insensitive CD8⁺ T cells. As a result, 1,25D3 can be beneficial in asthmatics, but restricted to a subpopulation of asthmatics characterized by the presence of these steroid-insensitive CD8⁺ T cells. Translating findings from the mouse model of experimental asthma, the inventors identified similar mechanistic links which contribute to the susceptibility to childhood asthma, thus providing new targets and novel therapeutic strategies in asthma, allergies and acute allergic responses.

The following examples are provided for illustrative purposes, and are not intended to limit the scope of the invention as claimed herein. Any variations which occur to the skilled artisan are intended to fall within the scope of the present invention. All references cited in the present application are incorporated by reference herein to the extent that there is no inconsistency with the present disclosure.

### EXAMPLES

### Methods

Animals: OT-1 mice expressing a transgenic T cell receptor (TCR) specific for SIINFEKL peptide (ovalbumin (OVA257-264) (Jia, Y., et al. Steroidogenic enzyme CYP11A1 regulates type 2 CD8⁺ T cell skewing in allergic lung disease. Proc. Natl. Acad. Sci. USA 110, 8152-8157 (2013); Jia, Y., et al. Stepwise epigenetic and phenotypic alterations poise CD8⁺ T cells to mediate airway hyperresponsiveness and inflammation. J. Immunol. 190, 4056-4065 (2013)) and homozygous CD8-deficient mice (Miyahara, N., et al. Effector CD8⁺ T cells mediate inflammation and airway hyper-responsiveness. Nat. Med. 10, 865-869 (2004); Ohnishi, H., et al. Corticosteroids enhance CD8⁺ T cell-mediated airway hyperresponsiveness and allergic inflammation by upregulating leukotriene B4 receptor 1. J. Allergy Clin. Immunol. 121, 864-871 e864 (2008)) were bred in the animal facility at National Jewish Health (Denver, Colo.). Studies were conducted under a protocol approved by the Institutional Animal Care and Use Committee of National Jewish Health.

CD8⁺ T cell culture: CD8⁺ effector memory T cells were generated in vitro as previously described (Jia, Y., et al. Steroidogenic enzyme CYP11A1 regulates type 2 CD8⁺ T cell skewing in allergic lung disease. Proc. Natl. Acad. Sci. USA 110, 8152-8157 (2013); Miyahara, N., et al. Effector CD8⁺ T cells mediate inflammation and airway hyper-responsiveness. Nat. Med. 10, 865-869 (2004); Jia, Y., et al. Stepwise epigenetic and phenotypic alterations poise CD8⁺ T cells to mediate airway hyperresponsiveness and inflammation. J. Immunol. 190, 4056-4065 (2013); Ohnishi, H., et al. Corticosteroids enhance CD8⁺ T cell-mediated airway hyperresponsiveness and allergic inflammation by upregulating leukotriene B4 receptor 1. J. Allergy Clin. Immunol. 121, 864-871 e864 (2008)). In brief, mononuclear cells (MNCs) were isolated from the spleens of OT-1 mice followed by stimulation with 1 μg/ml SIINFEKL (SEQ ID NO:1) peptide for 1.5 hours. Two days later, living cells were isolated using histopaque (Sigma-Aldrich, St. Louis, Mo.) and cultured in complete RPMI 1640 medium that contained recombinant mouse IL-2 (20 ng/ml, R&D, Minneapolis, Minn.) or IL-2+IL-4 (20 ng/ml, Peprotech, Rocky Hill, N.J.). For some experiments, the active form of Vitamin D3, 1α,25(OH)₂D₃ (referred to as 1,25D3, Sigma-Aldrich, St. Louis, Mo.) at a concentration of 100 nM or 1 μM was additionally added. Medium with cytokines was changed every day on 4 consecutive days. Cells were then re-stimulated with 1 μg/ml SIINFEKL (SEQ ID NO:1) in medium containing 2 μM monensin (Calbiochem, La Jolla, Calif.) for 4 hours. In some experiments, 100 nM or 1 μM 1,25D3 was additionally added to the medium during the antigen re-stimulation stage.

RNA Preparation and qPCR: Total RNA was extracted from 5×10⁶ differentiated CD8⁺ T cells using the NucleoSpin RNA II (Macherey-Nagel, Duren, Germany) isolation kit following the manufacture's protocol. Total RNA (1 μg) was converted into cDNA using QuantiTect reverse transcription kit (Qiagen, Valencia, Calif.). Specific primers and probes for qPCR of Gata3, Tbx21, Cyp11a1 , Vdr and the housekeeping gene 18SrRNA were designed with Vector NTI advance10 (Life Technologies, Carlsbad Calif.) and for Tbx21 a pre-designed assay (Mm00450960_m1, LifeTechnologies, Carlsbad, Calif.) was used (Table 1). The determined cycle threshold (Ct) reflects the number of PCR cycles required for the fluorescence signal to exceed the detection threshold, which was set to the log-linear range of the amplification curve. The differences in Ct values of the gene of interest and the house keeping gene 18SrRNA were used to calculate delta Ct (ΔCt). Relative fold changes (RFC) were then calculated using the 2^{-ΔΔCt} algorithm.

TABLE 1
qPCR primers used for gene expression experiments:

| Primer name | Sequence (5′3′) | SEQ ID NO: |
|---|---|---|
| Gata3 forward | GACTCTTCCCACCCAGCAGC | SEQ ID NO: 2 |
| Gata3 reverse | CCATCTCGCCGCCACAG | SEQ ID NO: 3 |
| Gata3 probe | CAAGGCACGATCCAGCACAG | SEQ ID NO: 4 |
| Tbx21 (LifeTechnologies | Mm00450960_m1 | — |
| pre-designed assay) | | |
| Cyp11a1 forward | TGATGACCTATTCCGCTTTTCC | SEQ ID NO: 5 |
| Cyp11a1 reverse | GGTTGAGCATGGGGACACTG | SEQ ID NO: 6 |
| Cyp11a1 probe | ATGCTGGAGGAGATCGTGGA | SEQ ID NO: 7 |
| Vdr forward | AGAAGGCTCCGATGACCCC | SEQ ID NO: 8 |
| Vdr reverse | AAGGTAAAAGACTGGTTGGAGCG | SEQ ID NO: 9 |
| Vdr probe | CCGCTCTCCATGCTGCCCCACC | SEQ ID NO: 10 |
| 18SrRNA forward | AGTCCCTGCCCTTTGTACACA | SEQ ID NO: 11 |
| 18SrRNA reverse | GATCCGAGGGCCTCACTAAAC | SEQ ID NO: 12 |
| 18SrRNA probe | CGCCCGTCGCTACTACCGATTGG | SEQ ID NO: 13 |

ELISA for Pregnenolone Measurements: Supernatants of differentiated CD8⁺ T cells were collected after culturing 5×10⁶/ml cells in 6-well plates for 24 hours with IL-2 or IL-2+IL-4 in the absence or presence of 1,25D3 (100 nM or 1 μM). Pregnenolone levels were measured using the pregnenolone ELISA kit (ALPCO Diagnostics, Salem, N.H.), as described previously ((Jia, Y., et al. Steroidogenic enzyme CYP11A1 regulates type 2 CD8⁺T cell skewing in allergic lung disease. Proc. Natl. Acad. Sci. USA 110, 8152-8157 (2013); Miyahara, N., et al. Effector CD8⁺ T cells mediate inflammation and airway hyper-responsiveness. Nat. Med. 10, 865-869 (2004); Jia, Y., et al. Stepwise epigenetic and phenotypic alterations poise CD8⁺ T cells to mediate airway hyperresponsiveness and inflammation. J. Immunol. 190, 4056-4065 (2013); Ohnishi, H., et al. Corticosteroids enhance CD8⁺ T cell-mediated airway hyperresponsiveness and allergic inflammation by upregulating leukotriene B4 receptor 1. J. Allergy Clin. Immunol. 121, 864-871 e864 (2008)).

Immunoblot Analyses: CD8⁺ T cells (5×10⁶) were lysed with RIPA buffer containing Halt™ protease and phosphatase inhibitor cocktail (Thermo Scientific, Rockford, Ill.) on ice for 30 minutes (Jia, Y., et al. Steroidogenic enzyme CYP11A1 regulates type 2 CD8⁺ T cell skewing in allergic lung disease. Proc. Natl. Acad. Sci. USA 110, 8152-8157 (2013); Jia, Y., et al. Stepwise epigenetic and phenotypic alterations poise CD8⁺ T cells to mediate airway hyperresponsiveness and inflammation. J. Immunol. 190, 4056-4065 (2013)). Samples were run by sodium dodecyl sulfate polyacrylamide gel electrophoresis (SDS-PAGE) and transferred to nitrocellulose membranes. The membranes were blocked using buffer containing 2% BSA and 0.5% sodium azide in TBST for 1 hour and incubated with rabbit polyclonal CYP11A1 antibody (Lifespan Biosciences, Seattle, Wash.) overnight at 4° C. Horseradish peroxidase-conjugated anti-rabbit IgG (GE Healthcare, Hertfordshire, UK) was used to detect CYP11A1 protein. Mouse monoclonal anti-β-actin antibody (Sigma-Aldrich, St. Louis, Mo.) was used as an internal control. Immunoreactive bands were quantified by densitometric quantification of autoradiographs using Image J (NIMH, Bethesda, Md.), and expressed as relative CYP11A1 normalized to β-actin (Sigma-Aldrich, St. Louis, Mo.).

Chromatin Immunoprecipitation (ChIP): ChIP assays of cultured CD8⁺ T cells (5×10⁶) with IL-2 or IL-2+IL-4 in the presence or absence of 100 nM or 1 μM 1,25D3 were performed according to manufacturer protocols using the ChIP IT express kit (Active Motif, Carlsbad, Calif.). Briefly, chromatin was cross-linked for 7 minutes at room temperature by adding 1% methanol-free formaldehyde (Thermo Fisher, Waltham, Mass.). Crosslinking was stopped by the addition of 1× glycine-stop fix solution at room temperature. Following incubation of cells for 30 minutes at 4° C., cell nuclei were pelleted and resuspended in 130 μl shearing buffer containing protease inhibitors. Shearing was conducted using an optimized 23-cycle treatment with a focused energy isothermal sonicator (Covaris S2 sonicator, Woburn, Mass.) leading to an average size of 300-500 base pairs, with highest density at 500 bp. Supernatant (10 μl) was saved for use as total input DNA.

Immunoprecipitations were performed according to manufacturer protocols with protein G magnetic beads overnight at 4° C. with an estimated 2 μg sheared chromatin. Equal amounts of chromatin were used for the immunoprecipitation with the VDR-specific antibody (ab3508, Abcam, Cambridge, Mass.). After washing and eluting the beads with respective kit buffers, reverse-crosslinking was performed for 15 minutes at 95° C. followed by a proteinase K treatment (10 μg/ml) for 1 hour at 37° C. The immunoprecipitated genomic DNA was purified using the Qiagen PCR purification kit (Qiagen, Valencia, Calif.) and analyzed via SYBR green (SYBR green Select Mastermix, Life Technologies, Carlsbad, Calif.) qPCR.

In silico transcription factor binding analyses were performed using MatInspector (Genomatrix, Munich, Germany) and Alibaba2.2 (www.gene-regulation.com) to identify putative VDR binding sites in the Cyp11a1 promoter region. Respectively, Cyp11a1 promoter-specific primers covering VDR binding sites were designed using the VectorNTI 11 software (Table 2). qPCR data were analyzed via the percent input methodology: (2̂(CT of total input−CT of specific IP))×100 and relative fold change to percent input ratios using CD8⁺ T cells stimulated with IL-2 as baseline.

TABLE 2
Cyp11a1 promoter specific primers used for VDR
ChIP experiments:

| Primer | | |
|---|---|---|
| name | Primer sequence (5′3) | SEQ ID NO: |
| VDR_1fwd | CTGGGGCTACACCGTGATTC | SEQ ID NO: 14 |
| VDR_1rev | CTTTTAAGATCCCCCTGCCTC | SEQ ID NO: 15 |
| VDR_2fwd | CTTGCTAGAACCCAGTGTAATGAAC | SEQ ID NO: 16 |
| VDR_2rev | AAGTTCACAGCGGTCCTCG | SEQ ID NO: 17 |
| VDR_3fwd | GCATTGATCCCAGAGAGGTTAAG | SEQ ID NO: 18 |
| VDR_3rev | CTGGTCCCATTGGCTTCTG | SEQ ID NO: 19 |
| VDR_ | GCTTCCTGAGTTGAGTTTTGTTATG | SEQ ID NO: 20 |
| 4 + 5fwd | | |
| VDR_ | CCATCCCTCCCTCCAAGC | SEQ ID NO: 21 |
| 4 + 5rev | | |
| VDR_ | TCGCCTGTCTCTGCCTCC | SEQ ID NO: 22 |
| 6 + 7fwd | | |
| VDR_ | TGAGTTCTAGTCCCCAGCAGC | SEQ ID NO: 23 |
| 6 + 7rev | | |

Flow Cytometry Analyses: For intracellular staining, 1×10 /ml cells were washed twice with PBS containing 1% BSA, stimulated with 1 μg/ml SIINFEKL in the presence of 2 μM monensin at 37° C. for 4 hours. After fixation with 4% paraformaldehyde (Electron Microscopy Sciences, Hatfield, Pa.) and permeabilization with 0.1% saponin (Sigma-Aldrich, St. Louis, Mo.), cells were washed twice with PBS containing 1% BSA, then incubated with anti-mouse CD16/CD32 (2.4G2, BD Bioscience, San Jose, Calif.) at 4° C. for 5 minutes, then stained with FITC labeled anti-mouse IFN-γ (XMG 1.2, eBioscience, San Diego, Calif.) and PE-labeled anti-mouse IL-13 (eBiol3A, eBioscience, San Diego, Calif.). Cell staining was monitored on a FACSCalibur (BD Bioscience, San Jose, Calif.) and analyzed using Flowjo software (Tree Star, Inc, Ashland, Oreg.) (Jia, Y., et al. Steroidogenic enzyme CYP11A1 regulates type 2 CD8⁺ T cell skewing in allergic lung disease. Proc. Natl. Acad. Sci. USA 110, 8152-8157 (2013); Miyahara, N., et al. Effector CD8⁺ T cells mediate inflammation and airway hyper-responsiveness. Nat. Med. 10, 865-869 (2004); Jia, Y., et al. Stepwise epigenetic and phenotypic alterations poise CD8⁺ T cells to mediate airway hyperresponsiveness and inflammation. J. Immunol. 190, 4056-4065 (2013).; Ohnishi, H., et al. Corticosteroids enhance CD8⁺ T cell-mediated airway hyperresponsiveness and allergic inflammation by upregulating leukotriene B4 receptor 1. J. Allergy Clin. Immunol. 121, 864-871 e864 (2008)).

Secondary Allergen Challenge Model and Adoptive Transfer: The experimental protocol for sensitization and challenge to OVA was performed as described previously ((Jia, Y., et al. Steroidogenic enzyme CYP11A1 regulates type 2 CD8⁺ T cell skewing in allergic lung disease. Proc. Natl. Acad. Sci. USA 110, 8152-8157 (2013); Miyahara, N., et al. Effector CD8⁺ T cells mediate inflammation and airway hyper-responsiveness. Nat. Med. 10, 865-869 (2004); Jia, Y., et al. Stepwise epigenetic and phenotypic alterations poise CD8⁺ T cells to mediate airway hyperresponsiveness and inflammation. J. Immunol. 190, 4056-4065 (2013).; Ohnishi, H., et al. Corticosteroids enhance CD8⁺ T cell-mediated airway hyperresponsiveness and allergic inflammation by upregulating leukotriene B4 receptor 1. J. Allergy Clin. Immunol. 121, 864-871 e864 (2008)) with some modifications. CD8-deficient mice were sensitized with 20 μg of OVA (Calbiochem, La Jolla, Calif.) emulsified in 2.25 mg of alum (AlumImuject; Pierce, Rockford, Ill.) on days 0 and 14 by intraperitoneal injection. Sensitized mice were challenged with 0.2% OVA for 20 minutes on days 28, 29, and 30 using an ultrasonic nebulizer (model NE-U07; Omron Healthcare, Kyoto, JP). To address the effect of 1,25D3 on CD8⁺ T cell-mediated AHR, CD8⁺ T cells (5×10⁶) generated in medium containing IL-2 without or with 1,25D3 (100 nM or 1 μM) were injected into naive or OVA-sensitized CD8-deficient mice intravenously on day 44. Two hours after cell transfer, naive and sensitized mice were re-challenged (secondary) with 1% OVA for 20 minutes by nebulization. Airway function was measured and samples were collected 48 hours after the secondary challenge. For some ChIP experiments, CD8⁺ T cells cultured in the presence of IL-2 without or with 1 μM 1,25D3 and adoptively transferred into sensitized and challenged recipients were isolated from the lungs. T cells were isolated from the lung after lung digestion (Jia, Y., et al. Stepwise epigenetic and phenotypic alterations poise CD8⁺ T cells to mediate airway hyperresponsiveness and inflammation. J. Immunol. 190, 4056-4065 (2013); Kanehiro, A., et al. Inhibition of phosphodiesterase 4 attenuates airway hyperresponsiveness and airway inflammation in a model of secondary allergen challenge. Am. J. Respir. Crit. Care Med. 163, 173-184 (2001)) with magnetic-activated cell sorting beads by positive selection using CD8⁺ (Ly-2) microbeads (Miltenyi Biotec, San Diego, Calif.) providing 99% CD8⁺T cells (Kanehiro, A., et al. Inhibition of phosphodiesterase 4 attenuates airway hyperresponsiveness and airway inflammation in a model of secondary allergen challenge. Am. J. Respir. Crit. Care Med. 163, 173-184 (2001)).

Assessment of Airway Function: Airway function was assessed as described previously (Miyahara, N., et al. Effector CD8⁺ T cells mediate inflammation and airway hyper-responsiveness. Nat. Med. 10, 865-869 (2004); Ohnishi, H., et al. Corticosteroids enhance CD8⁺ T cell-mediated airway hyperresponsiveness and allergic inflammation by upregulating leukotriene B4 receptor 1. J. Allergy Clin. Immunol. 121, 864-871 e864 (2008)) by measuring changes in airway resistance (RL) in response to increasing doses of inhaled methacholine (MCh, Sigma-Aldrich, St. Louis, Mo.). Data were presented as percentage change from the baseline RL values after saline inhalation. Baseline RL values were not significantly different among the various groups.

Bronchoalveolar Lavage (BAL) Fluid Analyses: After measurement of AHR, lungs were lavaged via the tracheal tube with 1 ml of HBSS. The supernatants were collected and IL-4, IL-5, and IL-13 (eBiosicence, San Diego, Calif.) levels were measured by ELISA as described previously ((Miyahara, N., et al. Effector CD8⁺ T cells mediate inflammation and airway hyper-responsiveness. Nat. Med. 10, 865-869 (2004); Ohnishi, H., et al. Corticosteroids enhance CD8⁺ T cell-mediated airway hyperresponsiveness and allergic inflammation by upregulating leukotriene B4 receptor 1. J. Allergy Clin. Immunol. 121, 864-871 e864 (2008)). Total leukocyte numbers were counted and differentiated as described previously ((Miyahara, N., et al. Effector CD8⁺ T cells mediate inflammation and airway hyper-responsiveness. Nat. Med. 10, 865-869 (2004); Ohnishi, H., et al. Corticosteroids enhance CD8⁺ T cell-mediated airway hyperresponsiveness and allergic inflammation by upregulating leukotriene B4 receptor 1. J. Allergy Clin. Immunol. 121, 864-871 e864 (2008)).

Lung Histology: Lungs were fixed in 10% formalin, and then embedded in paraffin. Paraffin sections (5-μm thick) were stained with periodic acid-Schiff (PAS). Mucus-containing goblet cells were quantified as previously described (Jia, Y., et al. Steroidogenic enzyme CYP11A1 regulates type 2 CD8⁺ T cell skewing in allergic lung disease. Proc. Natl. Acad. Sci. USA 110, 8152-8157 (2013); Jia, Y., et al. Stepwise epigenetic and phenotypic alterations poise CD8⁺ T cells to mediate airway hyperresponsiveness and inflammation. J. Immunol. 190, 4056-4065 (2013)).

Statistical Analyses for In Vitro and In Vivo Mouse Experiments: For outcome variables with multiple measures per mouse (either within or between treatments), linear mixed models were employed, using the best available covariance structure for repeated measures (see Supplementary Methods for more detail); for outcome variables with one measure per mouse, general linear models were used, allowing separate variances by treatment (i.e., weighted least squares). Pairwise comparisons were performed using t-tests derived from these models. Planned tests (5 for each outcome) for AHR, differential cell count, BAL cytokine measurements, and lung histology (PAS) included comparisons using sensitized and challenged CD8-deficient mice either before or after adoptive transfer of untreated CD8⁺ T cells as baseline. For gene expression, CYP11A1 Western blot, pregnenolone, and ChIP experiments, CD8⁺ T cells differentiated either with IL-2 alone or in the presence of IL-2+IL-4 were used as baseline for all pair-wise comparisons (7 tests for each outcome). The Benjamini-Hochberg (Benjamini Y, Hochberg Y. Controlling the False Discovery Rate: a practical and powerful approach to multiple testing. J Royal Stat Soc Ser B. 57 (1), 289-300 (1995)) procedure was used for the set of 5 or 7 comparisons within each outcome variable to help control for false-positive test results, using a false discovery rate (FDR) of 0.05. All outcome variables analyzed were based on at least 3 independent experiments (for more detail see Supplementary Methods). In vivo experiments including AHR were performed with 11-13 mice per treatment group. Results were expressed as the mean±SEM.

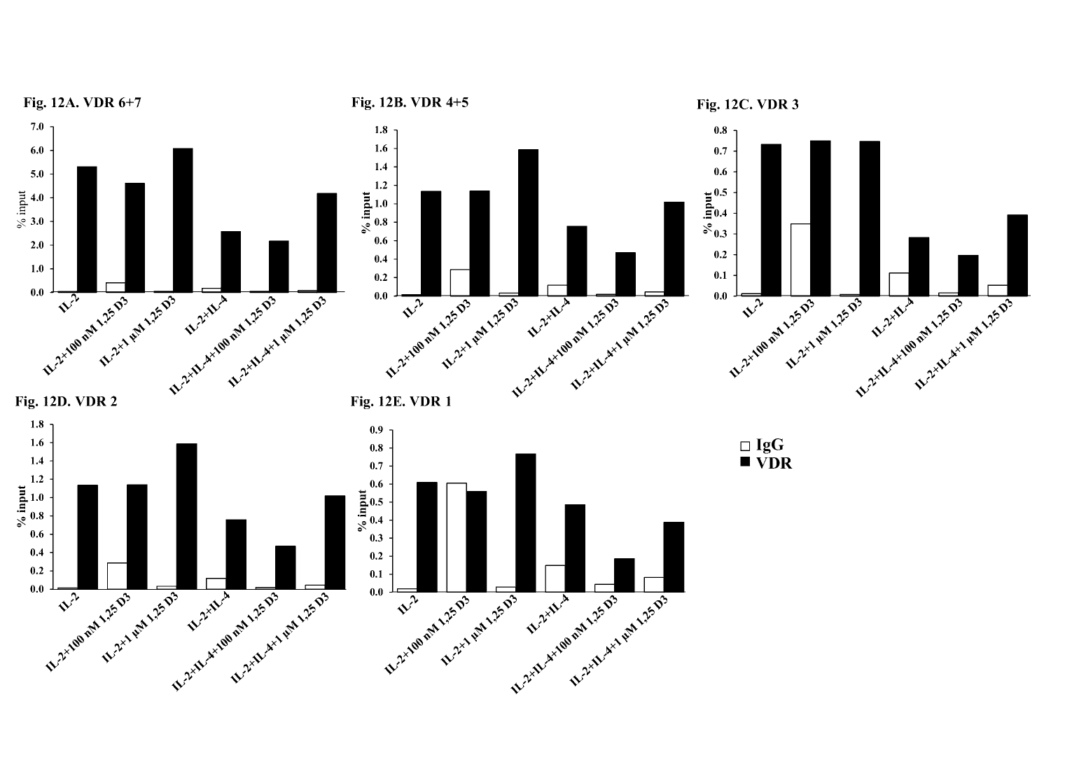

Human Study Populations and Genetic Analyses for CYP11A1 and VDR: Based on the linkage disequilibrium (r²≧0.8, HapMap database, release #28, CEU population) within the CYP11A1 locus (10 kb up- and 5 kb down-stream, FIGS. 12A-12E), 25 CYP11A1 SNPs were identified with a minor allele frequency (MAF) ≧0.03 and 2 SNPs leading to an amino acid change (rs6161 and rs1130841, MAF=0.005). These CYP11A1 polymorphisms clustered in 4 tagging bins and 4 single SNPs. Genotyping of rs9806234 failed but for the association analyses an imputation based dataset originated from HapMap II was available (Michel, S., et al. Unifying candidate gene and GWAS approaches in asthma. PloS One 5, e13894 (2010)). None of the SNPs deviated from Hardy-Weinberg Equilibrium (Table 1). No significant associations with asthma were observed for rs6161 (MAF=0.004, n=11 heterozygous subjects, 5 cases, 6 controls) and rs1130841 was monomorphic in the cohort under study. Thus, these mutations were not studied further.

Most of the asthmatics (n=655) for the association study included children of German or Austrian origin from the Multicenter Asthma Genetic In Childhood (MAGIC) study (mean age of 11±2.9years SD) (Michel, S., et al. Unifying candidate gene and GWAS approaches in asthma. PloS One 5, e13894 (2010); Moffatt, M. F., et al. Genetic variants regulating ORMDL3 expression contribute to the risk of childhood asthma. Nature 448, 470-473 (2007); Potaczek D P, et al. Different FCR1A polymorphisms influence IgE levels in asthmatics and non-asthmatics. Pediatr. Allergy Immunol. 24, 441-449 (2013)) and 73 asthmatics and 767 healthy controls were derived from the International Study of Asthma and Allergies in Childhood (ISAAC) Phase II (n=5,629, mean age of 9.6±0.6 years SD) (Weiland, S. K., et al. Prevalence of respiratory and atopic disorders among children in the East and West of Germany five years after unification. Eur. Respir. 1 14, 862-870 (1999)). As previously described (Potaczek D P, et al. Different FCR1A polymorphisms influence IgE levels in asthmatics and non-asthmatics. Pediatr. Allergy Immunol. 24, 441-449 (2013)), the subset of asthmatics was combined with asthmatics from the MAGIC study to test for associations in a case-control setting. No significant demographic differences between the populations under study were observed (Potaczek D P, et al. Different FCR1A polymorphisms influence IgE levels in asthmatics and non-asthmatics. Pediatr. Allergy Immunol. 24, 441-449 (2013). Genotyping of CYP11A1 was performed by Illumina HumanHap300Chip(rs2279357, rs11632698, rs2073475, rs4432229, N=1,311) (Michel, S., et al. Unifying candidate gene and GWAS approaches in asthma. PloS One 5, e13894 (2010); Moffatt, M. F., et al. Genetic variants regulating ORMDL3 expression contribute to the risk of childhood asthma. Nature 448, 470-473 (2007)) or by matrix-assisted laser desorption/ionization time-of-flight (MALDI-TOF) mass spectrometry (rs1484215, rs16968478, rs4432229, rs9806234, n=1,454, Table 3) (Michel, S., et al. Unifying candidate gene and GWAS approaches in asthma. PloS One 5, e13894 (2010); Schedel, M., et al. A signal transducer and activator of transcription 6 haplotype influences the regulation of serum IgE levels. J. Allergy Clin. Immunol. 114, 1100-1105 (2004)). Detailed information on the case-control dataset and genotyping methodology including oligonucleotide sequences are available from the authors upon request. Deviation from Hardy-Weinberg equilibrium was analyzed by chi-square test. Associations of binary traits were evaluated by logistic regression (Plink, version 1.07: pngu.mgh.harvard.edu/purcell/plink). Odds ratios (OR), 95% confidence intervals (CI) and p-values are reported. Adjustment for multiple testing for the association analyses for CYP11A1 SNPs was performed taking the LD into account. The effective number (Meff) of independent SNPs was calculated (SNPSpD (gump .qimr.edu.au/ general/daleN/SNPSpD) (Li J., et al. Adjusting multiple testing in multilocus analyses using the eigenvalues of a correlation matrix. Heredity. 95, 221-227 (2005)) to control the experiment-wise significance level with an adjusted p-value of p=0.010 (Li J., et al. Adjusting multiple testing in multilocus analyses using the eigenvalues of a correlation matrix. Heredity. 95, 221-227 (2005); Nyholt D R, A simple correction for multiple testing for single-nucleotide polymorphisms in linkage disequilibrium with each other. Am J Hum Genet. 74, 765-769 (2004)). Selection for SNPs within VDR for gene-by-gene-interaction analyses with CYP11A1 polymorphisms was based on previously studied VDR SNPs (Michel, S., et al. Unifying candidate gene and GWAS approaches in asthma. PloS One 5, e13894 (2010)).

TABLE 3
Asthma Associations for CYP11A1 SNPs in the case-control study population

| | | | | | | Asthma (n = 763) |
|---|---|---|---|---|---|---|
| | | | Call | | p-value | OR⁵ (95% CI) |
| SNP | Allele¹ | MAF² | rate (%) | LD³ bin | (HWE⁴) | p-value |
| rs2279357** | G/A | 0.28 | 99.7 | 5 | 0.64 | 0.85 (0.71-1.00) p = 0.0554 |
| rs6161 | G/A | 0.004 | 98.6 | | 1.00 | |
| rs11632698** | C/T | 0.40 | 99.9 | 4 | 0.42 | 0.85 (0.73-1.00) p = 0.0496 |
| rs1484215 | G/A | 0.08 | 98.1 | 3 | 0.58 | 1.00 (0.76-1.32) p = 0.9865 |
| rs1130841 | G/A | 0 | 98.3 | | | |
| rs2073475** | G/A | 0.14 | 98.9 | 2 | 0.26 | 0.98 (0.78-1.23) p = 0.8513 |
| rs16968478 | T/C | 0.17 | 97.6 | | 1.00 | 0.94 (0.77-1.14) p = 0.5293 |
| rs9806234^{#} | T/C | 0.26 | 100 | | 0.76 | 0.99 (0.84-1.18) p = 0.9478 |
| rs4886595 | T/G | 0.19 | 97.3 | | 0.91 | 0.77 (0.64-0.93) p = 0.0079^{&} |
| rs4432229** | T/C | 0.16 | 99.9 | 1 | 0.49 | 0.80 (0.65-0.98) p = 0.0351 |

¹Allele: wild-type/polymorphic allele.
²MAF: minor allele frequency.
³LD: linkage disequilibrium.
⁴HWE: Hardy Weinberg Equilibrium, p-value of χ²-test for deviation of HWE in controls.
⁵Odds ratios (OR) and 95% CI (confidence level): polymorphic (minor) allele used as basis for the calculation of the effect size.
^{#}Data obtained from imputed data set.
^{&}Association between rs4886595 and asthma remains significant after correction for multiple testing with an adjusted p-value of 0.010.
Significant associations in the case-control population (N = 1,454 or N = 1,311**) are marked in bold letters

In Silico Analyses of CYP11A1 SNPs: Functional annotation was performed of the tagging and tagged CYP11A1 SNPs associated with asthma. Transcription factor binding analyses depending on the genotype status were conducted by Matinspector (www.genomatix.de) and Alibab a (www.gene-regulation.com). The putative regulatory role was further evaluated by the regulomeDB (Boyle A P, et al. Annotation of functional variation in personal genomes using RegulomeDB, Genome Research. 22, 1790-1797 (2012)). The regulomeDB score and potential transcription role of histone modifications in naive CD8⁺ T cells isolated from peripheral blood is presented. Allele-specific analyses on CYP11A1 gene expression in EBV-transformed lymphoblastoid cell lines of unrelated samples of the HapMap population were performed as found atapp3.titan.uio.no/biotools.

### Example 1

This example shows that 1,25D3 prevents the functional conversion of CD8⁺ T cells from IFN-γ- to IL-13-producing cells.

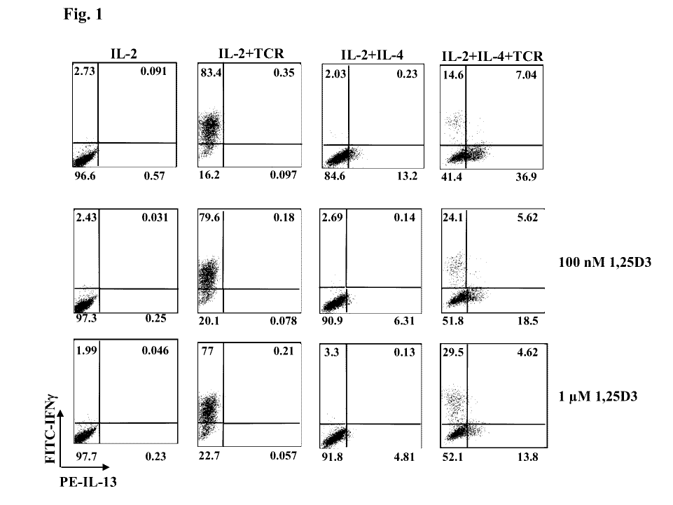

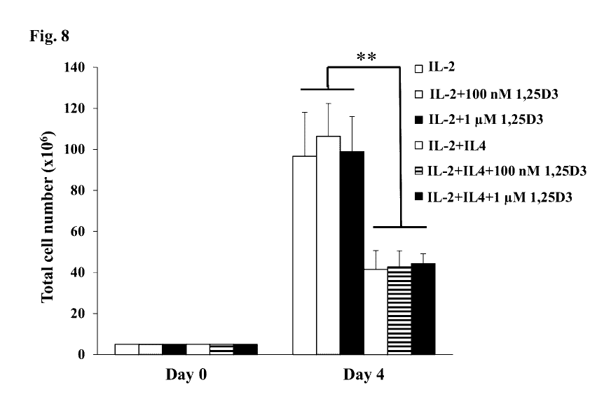

The inventors previously demonstrated that in the presence of IL-4, CD8⁺ T cells convert from IFN-γ CD8⁺ effector T cells to pathogenic IL-13-producers, triggering the full spectrum of lung allergic responses (Jia, Y., et al. Steroidogenic enzyme CYP11A1 regulates type 2 CD8⁺ T cell skewing in allergic lung disease. Proc. Natl. Acad. Sci. USA 110, 8152-8157 (2013); Jia, Y., et al. Stepwise epigenetic and phenotypic alterations poise CD8⁺ T cells to mediate airway hyperresponsiveness and inflammation. J. Immunol. 190, 4056-406 (2013)). To determine the effects of Vitamin D on this functional conversion of CD8⁺ T cells, the active form of Vitamin D, 1,25(OH)₂D₃ (further referred to as 1,25D3; 100 nM, 1 μM), was added during cell differentiation. 1,25D3 had no significant effect on cell viability (FIG. 8). When CD8⁺ T cells were cultured with SIINFEKL (SEQ ID NO:1) and IL-2+IL-4 in the presence of 1,25D3 a dose-dependent decrease in the percentage of IL-13⁺ cells and an increase in IFN-γ⁺ cells was observed (FIG. 1). After adding 100 nM 1,25D3, IL-13-single-positive cells decreased from 23.8±9.3% (mean±SEM) to 11.3±4.8% whereas IFN-γ-single-positive cells increased from 16.8±5.6% to 24.5±4.8% (FIG. 1, Table 4). This effect was even more pronounced after culture with 1 μM 1,25D3 (FIG. 1, Table 4).

TABLE 4
IFN-γ and IL-13 expression in CD8⁺ T cells differentiated in IL-2 or IL-2 + IL-4 in
the presence of 1,25D3

| | % of positive cells | | | | | |
|---|---|---|---|---|---|---|
| | | | | | IL-2 + | IL-2 + |
| | | IL-2 + | IL-2 + | | IL-4 + | IL-4 + |
| | | VitD | VitD | IL-2 + | VitD | VitD |
| | IL-2 | (100 nM) | (1 μM) | IL4 | (100 nM) | (1 μM) |
| IFN-γ⁺ | 3.3 ± 2 | 3.1 ± 1.3 | 4.3 ± 2.1 | 1.6 ± 0.8 | 2.2 ± 1 | 2.7 ± 1.2 |
| IL-13⁺ | 0.4 ± 0.2 | 0.2 ± 0.1 | 0.2 ± 0.1 | 9.7 ± 2.9 | 4.6 ± 1.1 | 3.8 ± 0.9 |
| IFN-ν⁺ | 0.1 ± 0.1 | 0 | 0 | 0.2 ± 0.2 | 0.2 ± 0.1 | 0.2 ± 0.2 |
| IL-13⁺ | | | | | | |
| IFN-ν⁻ | 96.2 ± 2 | 96.8 ± 1.3 | 95.8 ± 2.1 | 88.4 ± 3.2 | 92.7 ± 1.3 | 92.9 ± 1.3 |
| IL-13⁻ | | | | | | |

| | % of positive cells | | | | | |
|---|---|---|---|---|---|---|
| | | | | | IL-2 + | IL-2 + |
| | | IL-2 + | IL-2 + | | IL-4 + | IL-4 + |
| | | TCR + | TCR + | IL-2 + | TCR + | TCR + |
| | IL-2 + | VitD | VitD | IL-4 + | VitD | VitD |
| | TCR | (100 nM) | (1 μM) | TCR | (100 nM) | (1 μM) |
| IFN-γ⁺ | 79.8 ± 6.6 | 76.8 ± 5.3 | 76.2 ± 5.1 | 16.8 ± 5.6 | 24.5 ± 4.8 | 30.6 ± 5 |
| IL-13⁺ | 0.2 ± 0.3 | 0.1 ± 0.2 | 0.1 ± 0.1 | 23.8 ± 9.3 | 11.3 ± 4.8 | 8.3 ± 3.7 |
| IFN-ν⁺ | 0.7 ± 0.7 | 0.4 ± 0.5 | 0.4 ± 0.3 | 5.8 ± 1.9 | 4 ± 1.3 | 3.5 ± 1.2 |
| IL-13⁺ | | | | | | |
| IFN-ν⁻ | 20.1 ± 5.8 | 22.6 ± 4.3 | 23.7 ± 4.5 | 48.9 ± 6.9 | 55.1 ± 8.9 | 53.9 ± 6.5 |
| IL-13⁻ | | | | | | |

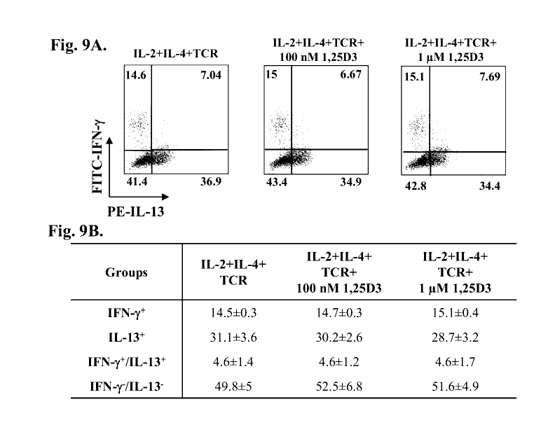

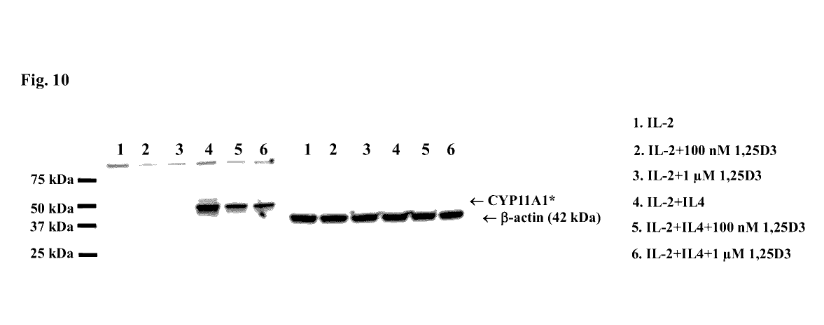

When 1,25D3 was added during the antigen (SIINFEKL) re-stimulation phase in the last 4 hours of culture, the cytokine profiles of differentiated CD8⁺ T cells generated in the presence of IL-2+IL-4 and 100 nM or 1 μM of the drug were unaffected (FIGS. 9A, 9B). These results suggested a significant role for 1,25D3 only during the conversion of CD8⁺ T cells in an IL-4-rich environment but not on differentiated cells.

### Example 2

This example shows that in CD8⁺ T cells, 1,25D3 alters functional activity of CYP11A1 (i.e., decreases CYP11A1 mRNA levels, protein levels and CYP11A1 enzymatic activity).

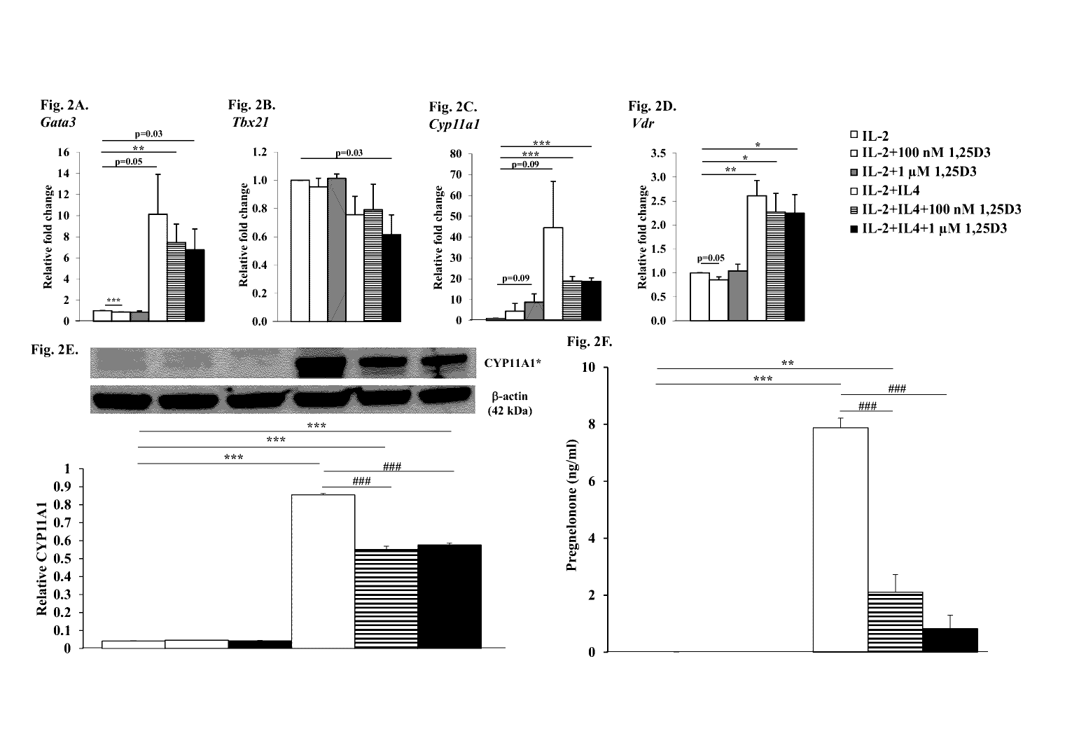

The major transcription factors, Tbx21 and Gata3, regulate expression of IFN-γ and IL-13 in T cells (Ngoc, P. L., Gold, D. R., Tzianabos, A. O., Weiss, S. T. & Celedon, J. C. Cytokines, allergy, and asthma. Curr. Opin. Allergy Clin. Immunol. 5, 161-166 (2005)). During the differentiation of CD8⁺ T cells with IL-2+IL-4, decreased Tbx21 mRNA levels were observed while Gata3 gene expression was increased compared to cells differentiated in IL-2 alone (FIGS. 2A, 2B). Along with changes in IFN-γ and IL-13 production, 1,25D3 affected Tbx21 and Gata3 gene expression only when present during CD8⁺ T-cell differentiation.

The enzymatic activation of CYP11A1 was previously demonstrated to play a key role in the phenotypic conversion of CD8⁺ T cells from IFN-γ- to IL-13-producing cells (Jia, Y., et al. Steroidogenic enzyme CYP11A1 regulates type 2 CD8⁺ T cell skewing in allergic lung disease. Proc. Natl. Acad. Sci. USA 110, 8152-8157 (2013); Jia, Y., et al. Stepwise epigenetic and phenotypic alterations poise CD8⁺ T cells to mediate airway hyperresponsiveness and inflammation. J. Immunol. 190, 4056-4065 (2013)). A similar mechanism during the stages of CD8⁺ T cell differentiation in the presence of 1,25D3 was thus studied. CYP11A1 transcript (FIG. 2C) and protein (FIG. 2E) levels were decreased in the presence of 1,25D3 (100 nM or 1 μM) during the differentiation of CD8⁺ T cells in IL-2+IL-4 to a greater extent than the lineage-specific transcription factors Gata3 and Tbx21. In parallel, decreased levels of pregnenolone were detected in supernatants from cells cultured in the presence of 1,25D3 (FIG. 2F). These data further support that in the presence of 1,25D3, CYP11A1 may be a functional regulator of the conversion of CD8⁺ T cells from IFN-γ- to IL-13-producing cells. In addition, in CD8⁺ cells differentiated in IL-2 +IL4, Vdr gene expression was significantly enhanced while additionally adding 1,25D3 during the differentiation process revealed a trend towards lower Vdr levels (FIG. 2D).

### Example 3

This examples that 1,25D3 alters VDR binding to the Cyp11a1 promoter.

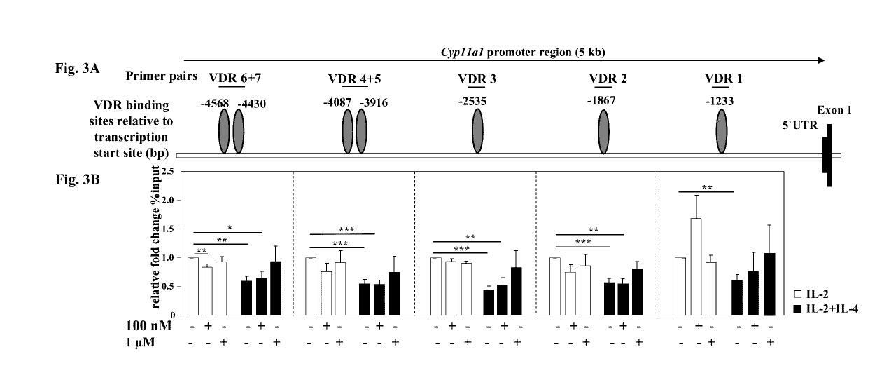

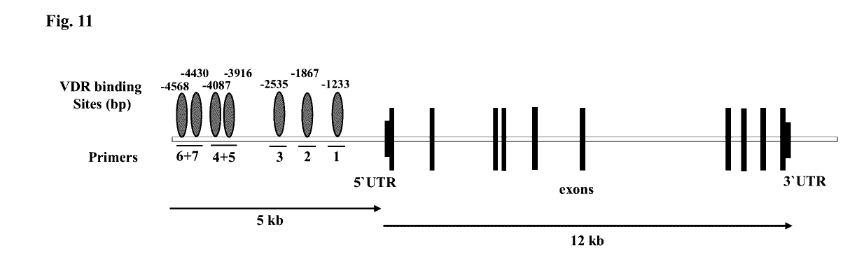

1,25D3 primarily mediates signals in the cell through the transcription factor Vitamin D receptor (VDR), which regulates the transcriptional activity of many target genes (Bosse, Y., et al. Asthma and genes encoding components of the Vitamin D pathway. Respir. Res. 10, 98 (2009); Wang, T. T., et al. Large-scale in silico and microarray-based identification of direct 1,25-dihydroxyvitamin D3 target genes. Mol. Endocrinol. 19, 2685-2695 (2005)). In silico transcription factor binding analyses of the Cyp11a1 promoter region (5 kb) predicted seven potential VDR binding sites (FIG. 11). To define the molecular mechanism underlying the 1,25D3-mediated prevention of the conversion of CD8⁺ T cells driven by CYP11A1, recruitment of VDR to the Cyp11a1 promoter region via chromatin immunoprecipitation (ChIP) was evaluated. In CD8⁺ T cells cultured in IL-2 or IL-2+IL-4 in the absence or presence of 1,25D3 (100 nM or 1 μM), the relative abundance of Cyp11a1 promoter DNA immunoprecipitated by a VDR-specific antibody was determined by qPCR using five Cyp11a1-specific primer pairs (FIG. 11, Supplementary Table 2). Due to the close vicinity of some of the predicted VDR binding sites, primers covered multiple potential VDR binding sites (e.g., primer pair 4 and 5, FIG. 11). A representative experiment of the percent input immunoprecipitated by the VDR and the negative control IgG antibody is presented in the FIGS. 12A-12E). Considering the complexity of the nuclear environment in primary CD8⁺ T cells, normalization between three independent experiments was performed via the percent input methodology and the relative percent input ratios were displayed using CD8⁺ T cells stimulated with IL-2 as baseline (as described more details in the Method section above). The data show that in the presence of IL-4 (black bars, FIG. 3B), the recruitment of VDR to the Cyp11a1 promoter region was significantly reduced for all VDR binding sites in comparison to CD8⁺ T cells stimulated with IL-2 alone (white bars, FIG. 3B). Surprisingly, CD8⁺ T cells cultured in IL-2+IL-4 together with 1,25D3 (1 μM) increased the association of VDR to Cyp11a1, almost to levels seen in IL-2-differentiated CD8⁺ T cells at baseline. In contrast, 1,25D3 had almost no effect on the VDR recruitment in CD8⁺ T cells after IL-2 stimulation alone. These results suggest that VDR binding to the Cyp11a1 promoter acts as a transcriptional repressor since the reduction in VDR recruitment to its promoter leads to increased CYP11A1 gene and protein expression (FIGS. 2C, 2E) and elevated pregnenolone levels (FIG. 2F).

### Example 4

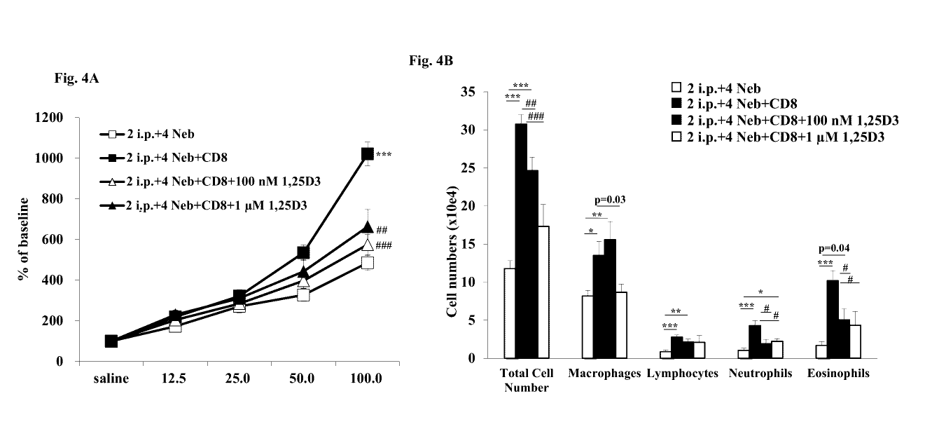

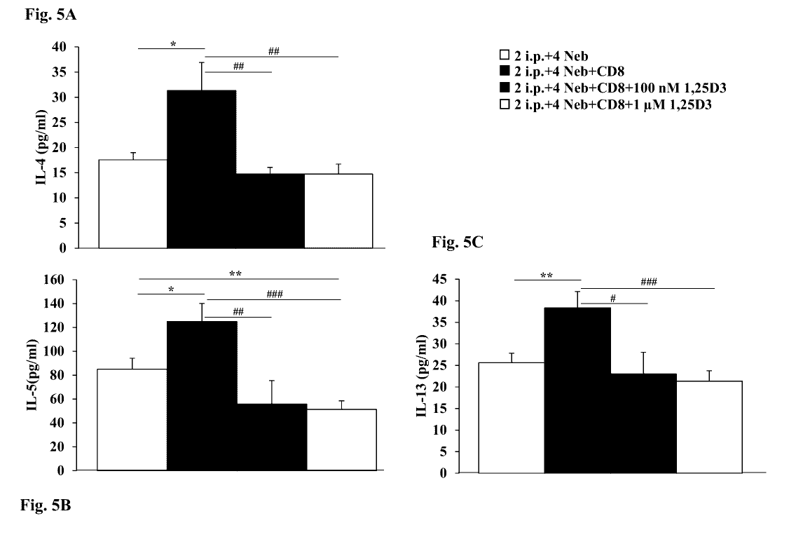

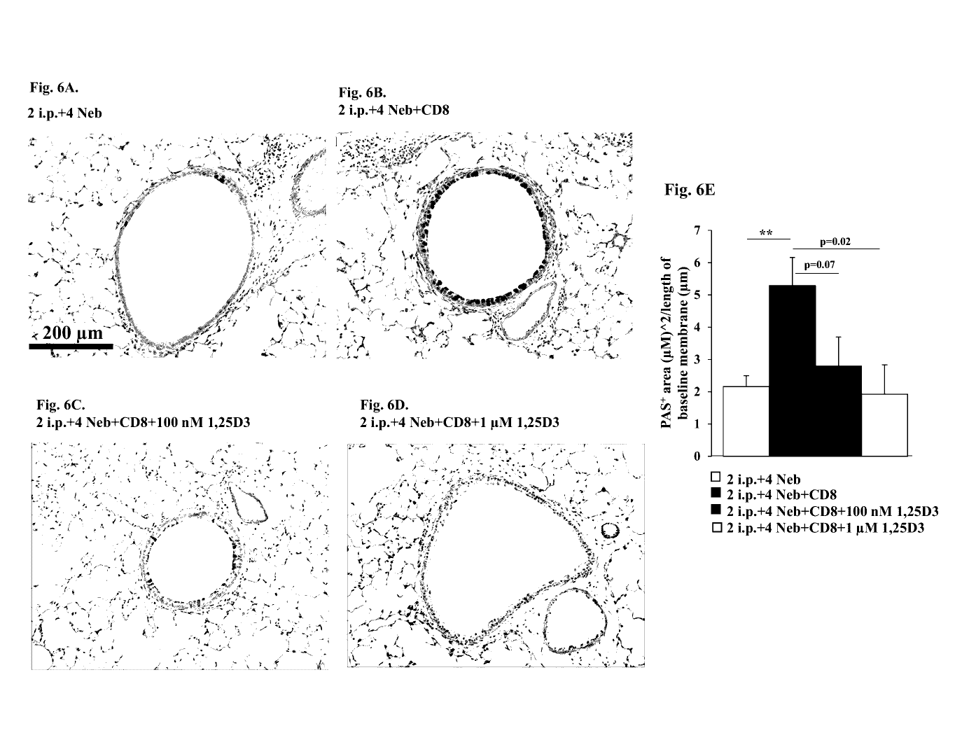

This example shows that 1,25D3-treated CD8⁺ T cells prevent lung allergic responses. Since 1,25D3 prevented the IL-4-induced conversion of CD8⁺ T cells from IFN-γ to IL-13 production in vitro, 1,25D3 treatment of CD8⁺ T cells was examined to determine if the treatment attenuated restoration of lung allergic responses following adoptive transfer in vivo into CD8-deficient mice. Adoptive transfer of IL-2-differentiated CD8⁺ T cells into sensitized and challenged CD8-deficient recipients followed by secondary allergen challenge fully restored lung allergic responses (FIGS. 4A, 4B). In contrast, transfer of IL-2-differentiated CD8⁺ T cells cultured in the presence of 1,25D3 (100 nM or 1 μM) failed to restore airway hyperresponsiveness (AHR) (FIG. 4A) or airway inflammation (FIG. 4B). Levels of IL-4, IL-5, and IL-13 were significantly lower in the bronchalveolar lavage (BAL) fluid of these recipient mice compared to recipients of untreated cells (FIGS. 5A-5C). Lung sections confirmed that these recipients showed less inflammation and significantly decreased numbers of PAS⁺ mucus-containing goblet cells (FIGS. 6A-6E).

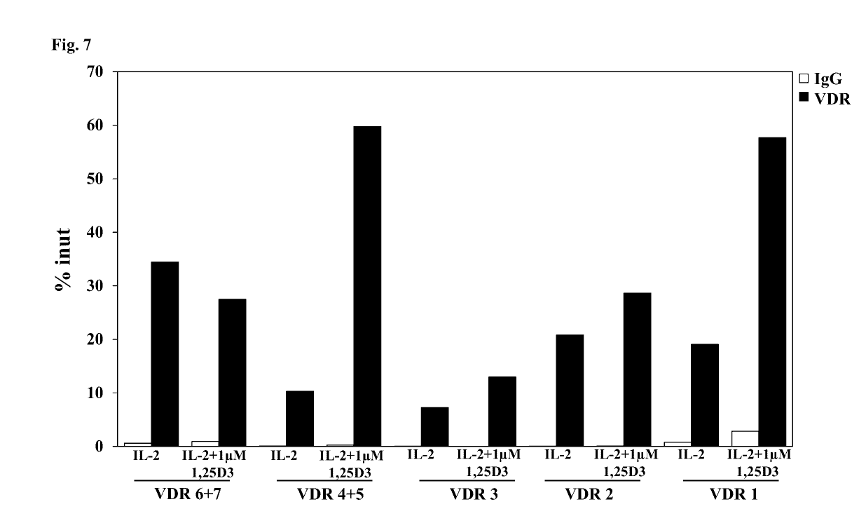

To confirm that the observed effects were VDR-dependent on the binding of VDR to the Cyp11a1 promoter region, adoptively-transferred CD8⁺ T cells were isolated from the lungs of ovalbumin (OVA)-sensitized mice after allergen challenge followed by ChIP experiments. As seen above in the in vitro experiments, isolated 1,25D3 treated CD8⁺ T cells from the lungs of sensitized and challenged recipients showed increased recruitment of VDR to the Cyp11a1 promoter region in comparison to recipients of untreated CD8⁺ T cells differentiated in IL-2 alone for four out of the five VDR binding sites under study (FIG. 7). These in vivo results together with the in vitro findings support that CYP11A1 plays a key role in both preventing the conversion of CD8⁺ T cells from IFN-γ- to IL-13-producers in the presence of 1,25D3 and the failure to induce AHR is the result of 1,25D3-triggered VDR binding to the Cyp11a1 promoter region.

### Example 5

This example shows genetic asthma predisposition of CYP11A1 in children.

Given the importance of CYP11A1 in the regulation of experimental asthma in mice, the inventors determined if a predisposition to asthma in humans was influenced by CYP11A1 single nucleotide polymorphisms (SNP). The 25 polymorphisms in CYP11A1 clustered in 5 tagging bins and 3 single SNPs were identified (Table 5). A significant protective effect in asthma was observed for three (rs4886595, rs4432229, rs11632698) out of eight tested SNPs in the case-control population with an odds ratio (OR) ranging from 0.77 to 0.85 (Table 1). Association results for SNP rs4886595 remained significant after correction for multiple testing (Table 1). Seven out of the eight polymorphisms covered by the asthma-associated tagging SNPs were located in potential regulatory regions of CYP11A1 (promoter: N=6, introns: N=1). Allele-specific in silico analyses predicted changes in transcription factor binding for all of these SNPs (Table 5). Computed score-based analyses of multiple high-throughput datasets (RegulomeDB Database, Table 6) suggested a putative functional differentiation protocol regulatory role of some of the asthma-associated CYP11A1 SNPs. Interestingly, for polymorphism rs8039957 (tagging bin 1, covered by rs4432229) located in the CYP11A1 promoter region, a novel VDR binding site was predicted in the presence of the polymorphic allele. Three CYP11A1 SNPs within this block and rs4886595 revealed allele-specific alterations on CYP11A1 gene expression (Table 6).

TABLE 5
Description of CYP11A1 polymorphisms and their respective rs numbers, position
within the gene, allele frequencies, linkage disequilibrium and genotyped tagging SNPs.

| | | | LD² (r²) | | |
|---|---|---|---|---|---|
| | Position in the | Minor allele | with | | Tagging |
| rs number | gene structure | frequency (MAF)¹ | tagging SNP | Tagging SNP | block |
| rs12917557 | promoter | 0.45 | 0.90 | rs11632698 | 4 |
| rs7163158 | promoter | 0.11 | 1.0 | rs4432229 | 1 |
| rs3825944 | promoter | 0.17 | 1.0 | rs2073475 | 2 |
| rs3803463 | promoter | 0.07 | 1.0 | rs1484215 | 3 |
| rs4432229 | promoter | 0.11 | | | 1 |
| rs4077581 | promoter | 0.28 | 0.92 | rs2279357 | 5 |
| rs4278698 | promoter | 0.11 | 1.0 | rs4432229 | 1 |
| rs8039957 | promoter | 0.11 | 1.0 | rs4432229 | 1 |
| rs4886595 | promoter | 0.14 | | | |
| rs9806234 | promoter | 0.27 | | | |
| rs16968478 | promoter | 0.21 | | | |
| rs16968477 | promoter | 0.16 | 0.89 | rs2073475 | 2 |
| rs2073475 | promoter | 0.18 | | | 2 |
| rs1130841 | exon 1 | 0.005 | | | |
| rs7174179 | intron 1 | 0.27 | 1.0 | rs2279357 | 5 |
| rs1843090 | intron 1 | 0.27 | 1.0 | rs2279357 | 5 |
| rs1484215 | intron 2 | 0.08 | | | 3 |
| rs12916123 | intron 2 | 0.27 | 1.0 | rs2279357 | 5 |
| rs11632698 | intron 2 | 0.37 | | | 4 |
| rs6161 | exon 5 | 0.005 | | | |
| rs2279357 | intron 8 | 0.27 | | | 5 |
| rs900798 | downstream | 0.30 | 0.80 | rs2279357 | 5 |
| rs2959002 | downstream | 0.27 | 0.97 | rs2279357 | 5 |
| rs2959003 | downstream | 0.30 | 0.80 | rs2279357 | 5 |
| rs900802 | downstream | 0.29 | 0.87 | rs2279357 | 5 |
| rs11635047 | downstream | 0.37 | 0.95 | rs11632698 | 4 |
| rs2279356 | downstream | 0.27 | 0.89 | rs2279357 | 5 |

¹Minor allele frequency (MAF) = based on the HapMap CEU population
²LD = linkage disequilibrium

TABLE 6
In silico analyses for putative regulatory functions for asthma-associated
CYP11A1 SNPs.

| Tagging | | | Major | Minor | Major | Minor | Regulome | | beta-score (p- |
|---|---|---|---|---|---|---|---|---|---|
| bin | SNP | Location | allele¹ | allele¹ | allele² | allele² | DM Score³ | Histone mods³ | value)⁴ |
| 1 | rs8039957 | promoter | SP1 | SP1 | 3 | 8 | 6 | weak | −0.04 |
| | | | | | | | | Repressed | (0.0780) |
| | | | | | | | | PolyComb | |
| | rs4278698 | promoter | SP1, Tra-1 | REV-Erb A | 5 | 4 | no data | no data | −0.04 |
| | | | | | | | | | (0.1380) |
| | rs4432229 | promoter | CEBPα | HNF1C | 4 | 6 | 4 | active TSS | −0.04 |
| | | | | | | | | | (0.0780) |
| | rs7163158 | promoter | REV-Erb | REV-Erb | 16 | 18 | 6 | weak | −0.06 |
| | | | A, COUP | A, COUP, | | | | transcription | (0.0223) |
| | | | | ARP1, | | | | | |
| | | | | SP1 | | | | | |
| | rs4886595 | promoter | CEBPα,- | CEBPα,- | 10 | 13 | 3a | quiescent/low | −0.06 |
| | | | β, Oct-1, | β, Oct-1 | | | | | (0.0028) |
| | | | NF-EM5, | | | | | | |
| | | | MyoD | | | | | | |
| 2 | rs12917557 | promoter | NF-κB, | NF-κB, | 6 | 7 | 5 | quiescent/low | −0.01 |
| | | | PTF1β, | PTF1β, | | | | | (0.6262) |
| | | | SP1 | GCN4, | | | | | |
| | | | | SP1 | | | | | |
| | rs1163269 8 | intron 2 | GR, | CEBPα | 13 | 17 | 2a | quiescent/low | −0.04 |
| | | | CEBPα | | | | | | (0.1380) |
| | rs11635047 | Down- | | | | | 5 | quiescent/low | 0.01 |
| | | stream | | | | | | | (0.7371) |

SNPs in bold letters indicate tagging SNPs
¹Different predicted transcription factor (TF) binding sites depending on the genotype using Matinspector (www.genomatix.de).
²Different predicted TF binding sites depending on the genotype using Alibaba (www.gene-regulation.com).
³Putative regulatory function based on regulomeDB, predicted histone modifications are based on data available from naive primary CD8⁺ cells from peripheral blood.
2a: TF binding + matched TF motif + matched DNase footprint + DNase peak
3a: TF binding + any motif + DNase peak
4: TF binding + DNase peak
5: TF binding + DNase peak
6: Other
⁴Allele-specific gene expression analyses in EBV-transformed lymphoblastoid cell lines of unrelated samples of the HapMap population (http://app3.titan.uio.no/biotools).

Because of these findings and the observed transcriptional regulation of Cyp11a1 by VDR in mice, a combined analysis of SNPs in CYP11A1 and VDR was performed to further determine if an epistatic phenomenon, here defined as the effect of one locus being dependent on the genotype of a second locus, is involved in the development of childhood asthma. The most significant asthma-associated SNPs in this population for VDR rs2107301 (Michel, S., et al. Unifying candidate gene and GWAS approaches in asthma. PloS One 5, e13894 (2010)) and CYP11A1 rs4886595 were selected for the analyses. Additionally, CYP11A1 SNP rs4432229 was included, since rs8039957 which is within the same tagging bin, is predicted to affect allele-specific binding of VDR to the CYP11A/ promoter region. Epistasis was observed when the effects of the respective protective alleles in CYP11A1 or VDR were studied after stratification for individuals carrying the corresponding SNP located in the other gene (Table 7). Both CYP11A1 SNPs, rs4886595 and rs4432229, were significantly associated with asthma only in individuals carrying homozygous asthma-risk alleles of rs2107301 (VDR). Thus, CYP11A1 polymorphisms may alter the pro-allergic function of CYP11A1 specifically in combination with modifier SNPs in VDR.

TABLE 7
Epistatic Effects on Asthma Susceptibility of Polymorphisms
in CYP11A1 and VDR.

| | | Reference | Odds ratio | |
|---|---|---|---|---|
| Effect of | In individuals carrying | allele¹ | (95% CI) | p-value |
| CYP11A1 | | G | 0.77 | 0.0079 |
| rs4886595 | | | (0.64-0.93) | |
| CYP11A1 | | C | 0.80 | 0.0351 |
| rs4432229 | | | (0.65-0.98) | |
| VDR | | A | 0.76 | 0.0010 |
| rs2107301 | | | (0.64-0.89) | |
| CYP11A1 | VDR rs2107301 GG (n = 660) | G | 0.71 | 0.0176 |
| rs4886595 | | | (0.54-0.94) | |
| CYP11A1 | VDR rs2107301 GA + AA (n = 608) | G | 0.82 | 0.1894 |
| rs4886595 | | | (0.62-1.10) | |
| VDR | CYP11A1 rs4886595 TT (n = 835) | A | 0.75 | 0.0082 |
| rs2107301 | | | (0.60-0.93) | |
| VDR | CYP11A1 rs4886595 TG + GG (n = 433) | A | 0.73 | 0.0372 |
| rs2107301 | | | (0.54-0.98) | |
| CYP11A1 | VDR rs2107301 GG (n = 678) | C | 0.73 | 0.0354 |
| rs4432229 | | | (0.54-0.98) | |
| CYP11A1 | VDR rs2107301 GA + AA (n = 624) | C | 0.90 | 0.4785 |
| rs4432229 | | | (0.67-1.21) | |
| VDR | CYP11A1 rs4432229 TT (n = 928) | A | 0.75 | 0.0051 |
| rs2107301 | | | (0.61-0.91) | |
| VDR | CYP11A1 rs4432229 TC + CC (n = 374) | A | 0.76 | 0.0970 |
| rs2107301 | | | (0.56-1.05) | |

The entire case-control study population was stratified for teh CYP11A1 polymorphisms rs4886595, and rs4432229 and rs2107301 (VDR) to investigate the SNP effect on asthma susceptibility of one SNP in relation to the other polymorphism. The statistical analyses was performed by logistic regession modeling additive effects
¹Allele: polymorphic (minor) allele was used as basis for the calculation of the effect size.

### Example 6

This example provides additional functional evidence that rs8039957and rs4886595—both located in the CYP11A1 promoter region—contribute to the observed protective effects in preventing the development of childhood asthma.

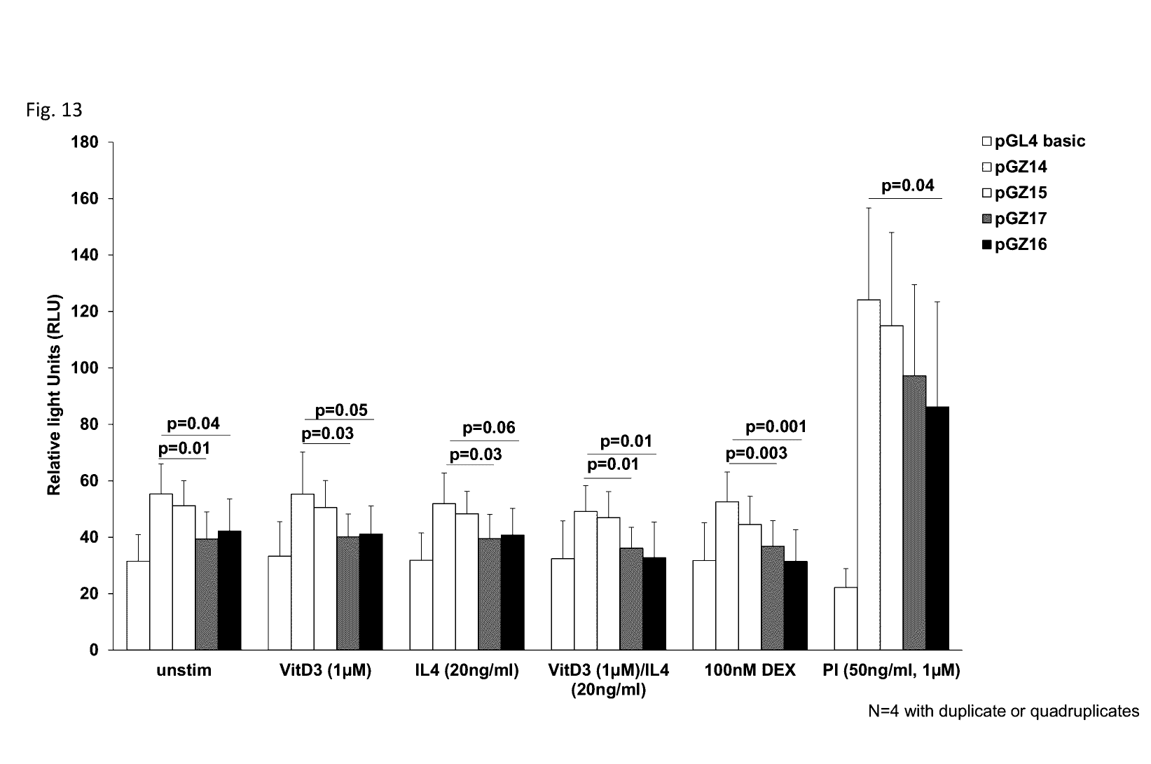

The allele-specific effects of rs8076131 and rs4065275 on CYP11A1 promoter activity were tested in a luciferase reporter system in HEK293 cells using pGL4 plasmids (Promega, Madison, USA) containing different combinations of the two SNPs in the CYP11A1 promoter region (pGZ14, pGZ15, pGZ17, pGZ16). The pRL-TK Renilla luciferase reporter plasmid (Promega, Madison, USA) was co-transfected for normalization of transfection efficiency and cell viability. Cells were in medium alone or stimulated (1 μM Vitamin D3, 20 ngml IL4, 1 μM Vitamin D3+20 ngml IL4, 100 nM dexamethasone, or 50 ng/ml PMA/1 μM ionomycin) 3 h after transfection and dual luciferase reporter assays were performed 21 h later. (FIG. 13)

Significant decreases in luciferase activity were observed in the presence of the polymorphic allele of rs8039957 (pGZ17) and the combination of both polymorphic alleles of rs8039957 and rs4886595 (pG16) before and after stimulation. In the presence of the polymorphic allele of rs4886595 (pGZ15), a similar trend was observed. The data indicate that both CYP11A1 SNPs contribute to CYP11A1 promoter activity.

In order to delineate if these changes in the CYP11A1 transcriptional activity were in fact due to changes in transcription factor binding, in silico analyses were performed. Importantly, the analyses indicated that in the presence of the CYP11A1 SNP rs8039957, an additional binding site for these transcription factor vitamin D receptor (VDR) was predicted while a loss of the binding of a glucocotricoid responsive element (GRE) for polymorphic allele of rs4886595 was also predicted. These findings suggest that the protective alleles (rs8039957, rs4886595) for the development of childhood asthma in the CYP11A1 promoter lead to lower CYP11A1 promoter activity due to changes of transcription factor binding of VDR and GRE.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

What is claimed:

1. A method of treating or preventing an allergic disease in a subject who has, or is at risk of developing an allergic disease, comprising administering to the subject a therapeutically effective amount of 1,25-dihydroxy vitamin D3 (1,25D3).

2. The method of claim 1, wherein the allergic disease is selected from the group consisting of an asthma, food allergy, allergic lung disease, allergen-induced airway hyperresponsiveness, allergen-induced inflammation, rhinitis, allergic rhinitis, eosinophilic esophagitis, chronic urticaria, atopic dermatitis, occupational allergy, allergic conjunctivitis, hay fever, airborne allergic sensitivities, stinging insect allergy, hypersensitivity pneumonitis, eosinophilic lung diseases, inflammatory bowel disease, ulcerative colitis, and Crohn's disease.

3. The method of claim 2, wherein the allergic disease is caused by one or more proteinaceous allergens.

4. The method of claim 1, wherein the subject has been sensitized to an allergen or is at risk of becoming exposed to an allergen.

5. The method of claim 2, wherein the food allergy is peanut allergy.

6. The method of claim 2, wherein the allergic disease is steroid-refractive asthma.

7. The method of claim 1, further comprising administering a therapeutically effective amount of a steroidogenic pathway inhibitor.

8. The method of claim 7, wherein the steroidogenic pathway inhibitor inhibits cytochrome P450 family 11 subfamily A polypeptide 1 (Cyp11A1).

9. The method of claim 8, wherein the steroidogenic pathway inhibitor is selected from the group consisting of aminoglutethimide, a Cyp11A1 siRNA and a Cyp11A1 shRNA molecule.

10. The method of claim 7, wherein the steroidogenic pathway inhibitor is trilostane or metyrapone.

11. The method of claim 1, wherein the subject is a human.

12. A method of preventing an acute allergic response in a subject in need thereof comprising administering to the subject a therapeutically effective amount of 1,25-dihydroxy vitamin D3 (1,25D3).

13. The method of claim 12, wherein the subject has an allergic disease selected from the group consisting of asthma, food allergy, allergic lung disease, allergen-induced airway hyperresponsiveness, allergen-induced inflammation, rhinitis, allergic rhinitis, eosinophilic esophagitis, chronic urticaria, atopic dermatitis, occupational allergy, allergic conjunctivitis, hay fever, airborne allergic sensitivities, stinging insect allergy, hypersensitivity pneumonitis, eosinophilic lung diseases, inflammatory bowel disease, ulcerative colitis, and Crohn's disease.

14. The method of claim 13, wherein the food allergy is peanut allergy.

15. The method of claim 13, wherein the allergic disease is steroid-refractive asthma.

16. The method of claim 12, further comprising administering a therapeutically effective amount of a steroidogenic pathway inhibitor.

17. The method of claim 16, wherein the steroidogenic pathway inhibitor inhibits Cyp11A1.

18. The method of claim 17, wherein the steroidogenic pathway inhibitor is selected from the group consisting of aminoglutethamide, a Cyp11A1 siRNA and a Cyp11A1 shRNA molecule.

19. The method of claim 16, wherein the steroidogneic pathway inhibitor is trilostane or metyrapone.

20. The method of claim 12, wherein the subject is a human.


SEQUENCE LISTING (23 sequences)

SEQ ID NO: 1 - protein, 8 aa, Artificial Sequence
  Synthetic peptide
SIINFEKL

SEQ ID NO: 2 - DNA, 20 bp, Artificial Sequence
  Synthetic primer; Gata3 forward
gactcttcccacccagcagc

SEQ ID NO: 3 - DNA, 17 bp, Artificial Sequence
  Synthetic primer; Gata3 reverse
ccatctcgccgccacag

SEQ ID NO: 4 - DNA, 20 bp, Artificial Sequence
  Synthetic primer; Gata3 probe
caaggcacgatccagcacag

SEQ ID NO: 5 - DNA, 22 bp, Artificial Sequence
  Synthetic primer; Cyp11a1 forward
tgatgacctattccgcttttcc

SEQ ID NO: 6 - DNA, 20 bp, Artificial Sequence
  Synthetic primer; Cyp11a1 reverse
ggttgagcatggggacactg

SEQ ID NO: 7 - DNA, 20 bp, Artificial Sequence
  Synthetic primer; Cyp11a1 probe
atgctggaggagatcgtgga

SEQ ID NO: 8 - DNA, 19 bp, Artificial Sequence
  Synthetic primer; VDR forward
agaaggctccgatgacccc

SEQ ID NO: 9 - DNA, 23 bp, Artificial Sequence
  Synthetic primer; VDR reverse
aaggtaaaagactggttggagcg

SEQ ID NO: 10 - DNA, 22 bp, Artificial Sequence
  Synthetic primer; VDR probe
ccgctctccatgctgccccacc

SEQ ID NO: 11 - DNA, 21 bp, Artificial Sequence
  Synthetic primer; 18SrRNA forward
agtccctgccctttgtacaca

SEQ ID NO: 12 - DNA, 21 bp, Artificial Sequence
  Synthetic primer; 18SrRNA reverse
gatccgagggcctcactaaac

SEQ ID NO: 13 - DNA, 23 bp, Artificial Sequence
  Synthetic primer; 18srRNA probe
cgcccgtcgctactaccgattgg

SEQ ID NO: 14 - DNA, 20 bp, Artificial Sequence
  Synthetic primer; VDR_1fwd
ctggggctacaccgtgattc

SEQ ID NO: 15 - DNA, 21 bp, Artificial Sequence
  Synthetic primer; VDR_1rev
cttttaagatccccctgcctc

SEQ ID NO: 16 - DNA, 25 bp, Artificial Sequence
  Synthetic primer; VDR_2fwd
cttgctagaacccagtgtaatgaac

SEQ ID NO: 17 - DNA, 19 bp, Artificial Sequence
  Synthetic primer; VDR_2rev
aagttcacagcggtcctcg

SEQ ID NO: 18 - DNA, 23 bp, Artificial Sequence
  Synthetic primer; VDR_3fwd
gcattgatcccagagaggttaag

SEQ ID NO: 19 - DNA, 19 bp, Artificial Sequence
  Synthetic primer; VDR_3rev
ctggtcccattggcttctg

SEQ ID NO: 20 - DNA, 25 bp, Artificial Sequence
  Synthetic primer; VDR_4+5fwd
gcttcctgagttgagttttgttatg

SEQ ID NO: 21 - DNA, 18 bp, Artificial Sequence
  Synthetic primer; VDR_4+5rev
ccatccctccctccaagc

SEQ ID NO: 22 - DNA, 18 bp, Artificial Sequence
  Synthetic primer; VDR_6+7fwd
tcgcctgtctctgcctcc

SEQ ID NO: 23 - DNA, 21 bp, Artificial Sequence
  Synthetic primer; VDR_6+7rev
tgagttctagtccccagcagc